(19) United States
(12) Patent Application Publication
(10) Pub. No.: US 2024/0415972 A1
(43) Pub. Date: Dec. 19, 2024

(54) MICROFRAGMENTED FAT TISSUE AS DRUG DELIVERY SYSTEM IN LIVER CANCER THERAPY

(71) Applicant: LIPOGEMS INTERNATIONAL S.P.A., Milano (IT)

(72) Inventors: Carlo Ferdinando Maria TREMOLADA, Milano (IT); Giulio ALESSANDRI, Milano (IT); Offer ZEIRA, Milano (IT); Augusto PESSINA, Milano (IT)

(21) Appl. No.: 18/704,227

(22) PCT Filed: Oct. 21, 2022
(86) PCT No.: PCT/EP2022/079388

§ 371 Date: Apr. 24, 2024

(30) Foreign Application Priority Data
Oct. 26, 2021 (IT) 102021000027437

(51) Int. Cl. A61K 47/46; A61K 9/00; A61K 31/337; A61K 31/44; A61K 31/675; A61K 31/704; A61P 35/00

(52) U.S. Cl. CPC A61K 47/46; A61K 9/0024; A61K 31/337; A61K 31/44; A61K 31/675; A61K 31/704; A61P 35/00

(57) ABSTRACT

The present invention refers to human micro-fragmented adipose tissue (MFAT) and its devitalized counterpart (DMFAT) loaded with proper anticancer agents for use in the treatment of liver cancer, preferably for use in the treatment of hepatocellular carcinoma (HCC).

### FIELD OF THE INVENTION

Liver cancer, namely hepatocellular carcinoma (HCC) poorly beneficiates from intravenous chemotherapy due to inadequate availability of drugs at tumor site. The present invention demonstrates the efficacy of human micro-fragmented adipose tissue (MFAT) and its devitalized counterpart (DMFAT) loaded with proper anticancer agents for use in the treatment of liver cancer, namely hepatocellular carcinoma (HCC).

### INTRODUCTION

Hepatocellular carcinoma (HCC) is one of the leading cancers in the world and, despite an improvement in therapeutic options in recent years, prognosis remains poor (1). Many factors contribute to the renown dismal overall survival, such as the low rate of patients that are suitable for a radical treatment due to a co-existing chronic liver disease and the high rate of recurrence observed after any type of treatment (2, 3, 4, 5). By now, no scheduled treatment is clearly defined and accepted to prevent tumor recurrence. Among possible therapeutic strategies, systemic therapies are widely implemented and studied, although typically characterized by low efficacy due to the impossibility of reaching effective concentrations of anticancer drugs specifically at tumor site due to systemic toxicity. For instance, the multi-kinase inhibitors (i.e. Sorafenib) can be used only with strict limitations in a narrow group of patients. For this reason, the development of strategies to increase chemotherapeutic agent delivering and specific localization at tumor site would be welcomed. Therapeutic approaches to increase localization of chemotherapy at the tumor site, reducing systemic toxicity are under deep investigation.

In previous studies the authors of the present invention discovered that adipose tissue (AT), after a process of fat microfragmentation (MFAT) became a natural scaffold able to delivery anti-cancer drugs (6). More specifically, it was found that fresh preparation of MFAT specimens through a specific process implemented by a device “Lipogems®” and, surprisingly, even its devitalized MFAT (DMFAT) counterpart, were very effective in adsorbing and releasing significant amount of chemotherapeutic agents, as the anti-cancer molecule Paclitaxel (PTX). Both MFAT and DMFAT loaded with PTX (MFAT-PTX; DMFAT-PTX) were able to kill many different human cancer cell lines in vitro when located nearby tumor cells, with an impressing long lasting anti-cancer activity. In addition, in vivo experiments focusing on DMFAT-PTX activity showed that in nude mice, orthotopically transplanted with Neuroblastoma (NB) cells and undergoing tumor surgical resection, the local application of DMFAT-PTX blocked or delayed tumor relapse (6). These results were the first demonstration that DMFAT may represent a very innovative natural biomaterial, able to localize and release anti-cancer molecules at the tumor site. Such drug-delivery approaches that can selectively deliver therapeutic drugs into tumor sites have demonstrated a great potential in cancer treatment as a tool to resolve the limitations of conventional chemotherapy. Numerous preclinical studies have been published, but targeted drug delivery systems for HCC have yet to be made for practical clinical use.

Based on such observations, since rational targeted drug delivery systems should take cancer-specific properties into consideration, the authors of the present invention studied the properties of HCC aiming to understand if the MFAT and DMFAT systems could be meaningful platform for development of HCC targeted therapies. Therefore within the present invention it was found a very high anticancer activity of DMFAT-PTX against a well-developed HCC primary tumor in in vitro and in vivo models. Moreover, inventors investigated if a single-shot application of DMFAT-PTX located nearby a tumor mass could be sufficient to inhibit or delay tumor growth, quantifying and evaluating the length of the eventual anti-tumor effect. The invention demonstrates for the first time that both fresh MFAT and its devitalized counterpart, DMFAT, loaded with PTX are very effective in inhibiting in vitro the growth of the HCC tumor cell line Hep-3B. In vivo experiments revealed that a single-shot administration of DMFAT-PTX placed near the HCC tumor site produces a potent growth inhibition effect on tumor cells.

Therefore the invention demonstrates for the first time that both fresh MFAT and DMFAT loaded with PTX are very effective in inhibiting the growth in vitro of liver cancer cells and in vivo DMFAT-PTX, placed in situ, blocked HCC in an advanced stage of growth, suggesting it as a new potent and viable delivery-drug system, for very aggressive cancers. DMFAT can be a potent and valid new tool for the local chemotherapy of HCC in an advanced stage of progression, suggesting a potential effectiveness also in other human liver cancer. This is of particular relevance as HCC poorly beneficiates from intravenous chemotherapy due to inadequate availability of drugs at tumor site. Starting from evidences demonstrating that human MFAT and its devitalized counterpart DMFAT are effective natural scaffolds to delivery Paclitaxel (PTX) to tumors in both in vitro and in vivo tests, affecting cancer growth, the present invention demonstrates the efficacy of DMFAT-PTX in a well-established HCC in nude mice.

### SUMMARY OF THE INVENTION

Within the present invention preparations of human micro-fragmented adipose tissue (MFAT) and its devitalized counterpart (DMFAT), loaded with Paclitaxel (MFAT-PTX and DMFAT-PTX) were evaluated for anti-cancer activity in 2D and 3D assays with Hep-3B tumor cells. In mice, efficacy of DMFAT-PTX was evaluated after a single-shot subcutaneous injection nearby a Hep-3B growing tumor by assessing tumor volumes, apoptosis rate and drug pharmacokinetic.

A potent antiproliferative activity was seen in both in vitro 2D and 3D tests. Mice treated with DMFAT-PTX (10 mg/kg) produced a potent Hep-3B growth inhibition with 33% of complete tumor regressions. All treated animals experimented a tumor ulceration at the site of DMFAT-PTX injection, which healed spontaneously. Lowering drug concentration (5 mg/kg) prevented ulcers formation maintaining a statistically significant efficacy against Hep-3B growth. Histology revealed a higher number of apoptotic cancer cells intratumorally, suggesting the prolonged presence of PTX that was confirmed by the pharmacokinetic analysis of the drug diffusion from fat tissue to cancer cells.

It is therefore an object of the invention a tissue-based drug delivery system or a composition comprising:
micro-fragmented fat tissue (MFAT) preferably comprising clusters of fat tissue having size ranging from 10 to 5000 μm, preferably from 100 to 3000 μm, more preferably from 200 to 2500 μm, still more preferably from 30 to 1500 μm, more preferably from 400 to 900 μm; and
at least one anticancer agent;
   for use in the treatment and or prevention of liver cancer, preferably hepatocellular carcinoma.

In a preferred embodiment in the above tissue-based drug delivery system the MFAT is devitalized micro-fragmented fat tissue (DMFAT).

Preferably the fat tissue is isolated from a mammalian, more preferably is isolated from humans said humans being alive or cadaver; still preferably said fat tissue is autologous or heterologous.

In a preferred embodiment of the invention the tissue-based drug delivery system comprises as the anticancer agent, an agent selected from a taxane, preferably paclitaxel or docetaxel, lenvatenib gemcitabine, mitomycin C, vinorelbine, vincristin, vinblastin, nocodazole, epothilones, navelbine, epidipodophyllotoxins, preferably teniposide, actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorethamine, mitomycin, mitoxantrone, nitrosourea, plicamycin, procarbazine, triethylenethiophosphoramide, etoposide (VP16), adriamycin, amsacrine, camptothecin, daunorubicin, dactinomycin, doxorubicin, eniposide, epirubicin, idarubicin, irinotecan (CPT-1 1) and mitoxantrone, pemetrexed, 5-flurouracile (5-FU), metotrexate, cyclophosphamide, bortezomib, tomozolomide, sorafenib or any combination thereof.

Preferably, the anticancer agent is a combination of:
sorafenib and paclitaxel; or
cyclophosphamide and paclitaxel; or
adriamycin and paclitaxel.

Even more preferably, the anticancer agent is paclitaxel or derivatives thereof.

Still preferably, in the tissue-based drug delivery system of the invention the amount of the anticancer agent ranges from 0.1 to 1 mg per ml of MFAT or DMFAT, or from 0.001 to 1 mg per ml of MFAT or DMFAT.

According to a further preferred embodiment, the amount of Paclitaxel or derivatives thereof, preferably docetaxel, for obtaining an anticancer effect/activity is not less than 150 ng for 100 μl of MFAT or DMFAT and/or not less than 300 ng for 100 μl of MFAT or DMFAT.

Nevertheless, the amount of the anticancer agent that can be loaded as maximum depends on the lipo-hydrophylic nature of said agent and on its solubility in the MFAT or DMFAT sample. Preferably the anticancer agent can be loaded on the MFAT or DMFAT specimens up to the relative saturation point.

In a preferred embodiment, the tissue-based drug delivery system of the invention is locally injected at the tumor tissue site and releases in situ a therapeutically effective amount of the anticancer agent.

As indicated above, the tissue-based drug delivery system of the invention is for use in the treatment and or prevention of liver cancer, preferably hepatocellular carcinoma, preferably said cancer is a liver primary or metastatic cancer, preferably primary or metastatic hepatocellular carcinoma.

It is a further object of the invention the tissue-based drug delivery system of the invention in combination with a further therapeutic treatment.

It is a further object of the invention a pharmaceutical formulation comprising the tissue-based drug delivery system according to anyone of previous claims and a pharmaceutically acceptable vehicle or excipient, preferably said pharmaceutical formulation is for use in the treatment and or prevention of liver cancer, preferably hepatocellular carcinoma.

The tissue-based drug-delivery system of the invention is based on micro-fragmented fat tissue as discussed in detail below.

As used herein, the term “tissue-based drug delivery system” indicates a composition comprising the micro-fragmented fat tissue as herein disclosed and at least one anticancer agent. The composition preferably comprises also saline.

Therefore, in the context of the present invention, the micro-fragmented fat tissue (MFAT), is used as a scaffold to deliver (a delivery system) high amount of anticancer drugs, such as Paclitaxel (PTX) or Sorafenib, or any related derivative, to cancer cells in the treatment of hepatocellular carcinoma.

The invention further demonstrates that devitalized MFAT, i.e. DMFAT, is very effective in absorbing and realizing the anticancer molecules in the context of the treatment of liver cancer. Said DMFAT can be obtained according to published procedures (6) based on freeze/thawing steps.

The tissue-based system of the invention allows molecules and/or drugs administration in individuals (any animal) in need thereof, wherein the molecules/drugs are delivered in the tumor site.

In the contest of the present invention, fat tissue means adipose tissue. Preferably, said fat tissue is isolated from any animal, more preferably it is isolated from a humans said humans being alive or a cadaver.

Preferably, said fat tissue derives/is isolated (purified) from any part of the body, preferably from the lower and/or the lateral abdomen area. Preferably, said fat tissue is isolated from the body by lipoaspiration/liposuction (lipoaspirate) procedure. Therefore, according to a preferred embodiment the fat tissue is a lipoaspirate (LASP) or derivatives thereof. In the context of the present invention, lipoaspiration or liposuction or simply lipo means the removal of adipose tissue (fat) under negative pressure condition, generally by using a cannula.

As already mentioned before, the fat tissue, preferably the lipoaspirate, is micro-fragmented (LPG in the example and drawings as an example of micro-fragmented fat tissue).

Preferably, the fat tissue is micro-fragmented by a non-enzymatic procedure and therefore the fat of the present invention is more preferably non-enzymatic micro-fragmented fat. In other words, the fat used/administered in the present invention as delivery system has been micro-fragmented without any enzymatic treatment.

According to the invention, the micro-fragmented fat tissue is obtained by using an innovative process disclosed in WO2018/193413, also implemented by the Lipogems device, more preferably according to the procedure as fully disclosed in the patent application WO2011/145075.

The fat tissue, preferably the lipoaspirate, is introduced in the Lipogems® device wherein it is progressively reduced (fragmented) in small clusters of fat tissue preferably by means of mild mechanical forces and, more preferably, in presence of a solution, preferably a saline solution.

According to a preferred embodiment, the micro-fragmented fat of the invention contains clusters of fat tissue having size ranging preferably from 10 to 5000 μm, more preferably from 100 to 3000 μm, still more preferably from 200 to 2500 μm, more preferably from 300 to 1500 μm, more preferably from 200 to 900 μm, more preferably from 200 to 800 μm, more preferably from 400 to 900 μm.

According to a further preferred embodiment, the fat, preferably the micro-fragmented fat or the clusters of micro-fragmented fat, comprise Mesenchymal Stem Cells (MSCs) and/or Adipose-derived Stem Cells (ASCs) and/or Adipose Stem Cells and/or pericytes and/or adipocytes and/or endothelial cells. In this regard, particularly advantageous are the micro-fragmented fat clusters since they keep the natural/intact stromal vascular niche of the resident cells that, consequently, are supported by the stroma resembling the natural/physiological context in trophic and/or signaling terms. Additionally, the stroma provides a protected environment during the graft of the cells against any physical and/or chemical insults, such as mechanical, oxygen, ecc. Fat fragmentation inside the device is preferably controlled by using one or more fragmentation/disaggregation/emulsifying means. According to a preferred embodiment, said means are metallic means, more preferably metallic beads and/or filters/nets, wherein the filters/nets provide preferably a micro-fragmentation of the tissue sample, while the beads freely move inside the device in order to promote the separation between the solid part and the liquid part of the tissue sample and (inherently) provide an emulsion of the liquid parts with the a washing fluid. Preferably the beads have size (average diameter) ranging preferably from 0.1-30 millimeters, more preferably 1-20 mm, still more preferably 5-10 mm, still more preferably 7.5-8.5 mm and/or said filter/nets have average diameter ranging from 2000 μm to 200 μm, preferably from 1500 μm to 500 μm. The mesh average diameter (pore size) of the filter/net ranges between 50 μm and 6000 μm, preferably between 500 μm and 3000 μm.

It is advisable to perform mild movements of the fragmentation/disaggregation/emulsifying means throughout the fat tissue, more preferably by performing a controlled shaking of the device.

According to a preferred embodiment, the fragmentation/disaggregation/emulsification is performed in immersion, preferably with a continuous flow of saline buffer through the device, so allowing an easy washing of the tissue sample (in particular an effective oil and/or blood residues removal). More preferably, the fragmentation/disaggregation/emulsification is performed by washing the tissue sample through a continuous flow of the saline buffer that, together with beads shaking, allows the solid material to lift towards the inlet of the saline buffer, leaving the oil and/or blood residues to flow together with the saline towards the outlet.

The fragmentation/disaggregation/emulsification procedure lasts for preferably few seconds. Therefore, the micro-fragmented fat of the present invention is obtained by using a gentle, enzyme-free, sterile, intra-operative and rapid manipulation.

The fat tissue of the present invention is preferably isolated from any animal, more preferably from humans. Preferably said animal/human is healthy or cadaver. According to a preferred embodiment, the fat is animal adipose tissue, more preferably human adipose tissue, more preferably isolated/lipoaspirate from the lower and/or the lateral abdomen area of an individual. However, said fat can be isolated from any useful body area. Preferably, the micro-fragmented fat is autologous or heterologous.

In the contest of the present invention, the molecule to be delivered means any drug and/or prodrug or therapeutic substance used in the treatment of liver cancer or hepatocellular carcinoma. Preferably said molecule is lipophilic (poor water-soluble or water insoluble).

However, the tissue-based system of the invention is also suitable to deliver hydrophilic molecules/drugs. For the purpose of the present invention, the preferred molecules to be delivered are anti-cancer molecules (chemotherapeutics) preferably selected from: natural products, preferably vinca alkaloids, more preferably selected from: vinblastine, vincristine, and vinorelbine, taxane, preferably paclitaxel or docetaxel, vincristin, vinblastin, nocodazole, epothilones and navelbine, epidipodophyllotoxins (teniposide), actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorethamine, mitomycin, mitoxantrone, nitrosourea, plicamycin, procarbazine, teniposide, triethylenethiophosphoramide and etoposide (VP16)), adriamycin, amsacrine, camptothecin, daunorubicin, dactinomycin, doxorubicin, eniposide, epirubicin, etoposide, idarubicin, irinotecan (CPT-1 1) and mitoxantrone, pemetrexed, 5-FU, rafenib, metotrexate, cyclophosphamide, bortezomib, tomozolomide, sorafenib. Any combination of the molecules reported above should be considered forming part of the present disclosure. More preferably, the molecules are selected from: Paclitaxel (PTX—Taxol or Onxal) or derivatives thereof, preferably selected from Abraxane and/or Docetaxel, doxorubicin or derivative thereof, preferably Adriamycin and/or, Vincristine and any combination thereof. Still preferably the molecule is Sorafenib.

The anti-cancer molecules can be delivered also in combination with further molecules, preferably selected from: antibiotics, anti-inflammatory substances, poli- or mono-clonal antibodies, immunomodulatory molecules, biological drugs and combinations thereof. The molecule and/or the drug/prodrug can be modified in any way, such as pegylation or it can be associated with particles, preferably nanoparticles, such as albumin-nanoparticles.

The tissue-based delivery system of the invention, loaded/primed with molecules and/or drugs as disclosed above, is used for the targeted treatment of liver cancer, preferably for the treatment of hepatocellular carcinoma. Targeted drug delivery systems that can selectively deliver therapeutic drugs into the tumor site are not available for practical clinical use, particularly for hepatocellular carcinoma.

According to a preferred embodiment, the amount said molecules/drugs that can be loaded/primed into the delivering system of the invention ranges from 0.5 to 10 mg/ml of fat tissue.

According to a further preferred embodiment, the amount of Paclitaxel (PTX) or derivatives thereof, preferably Abraxane, Docetaxel, for obtaining an anti-cancer effect/activity is not less than 150 ng for 100 ul of micro-fragmented fat tissue/lipoaspirate (LPG). Nevertheless, the amount that can be loaded as maximum depends on the lipo-hydrophylic nature of the drug.

The amount of the molecules/drugs released per day by the delivery system of the invention ranges from 10-15% compared to the loading/priming amount that is the amount used to prime the micro-fragmented fat tissue/lipoaspirate (LPG).

According to a preferred embodiment of the invention, the tissue-based delivery system is for local, parenteral, peritoneal, mucosal, dermal, epidermal, subcutaneous or transdermal, administration.

When the tissue-based delivery system of this invention is administered into a subject, the volume dosage and the frequency of administrations will normally be determined by the prescribing physician according to the age, weight, sex and response of the individual patient, as well as tumor mass volume and severity of the patient's symptoms. Preferably the tissue-based delivery system of the invention wherein the concentration of active drug is comprised between 100 to 1000 μg/ml of MFAT or DMFAT, is administered to the patient in an amount comprised between 5 to 30 ml, preferably 7 to 20 ml.

According to a further preferred embodiment, the tissue-based delivery system of the invention, eventually loaded with the molecules/drugs as disclosed above, is administered/applied in combination (pre-post) radiotherapy and/or surgery. Preferably the tissue-based delivery system of the invention, eventually loaded with the molecules/drugs as disclosed above, is applied on the interested area before surgery for example in order to reduce the tumor area to be removed and therefore, to make the surgery less traumatic. The tissue-based delivery system of the invention, eventually loaded with the molecules/drugs as disclosed above, is preferably pre and/or post-operatory administration/application, preferably topical, intraperitoneal, subcutaneous, administration/application, preferably for preventing the cancer relapses, more preferably for metastatic tumors.

### FIGURES LEGEND

FIG. 1. MFAT and DMFAT specimens loaded with different PTX concentrations displayed a dose-dependent anti-cancer activity on human Hep-3B cancer cells in vitro.

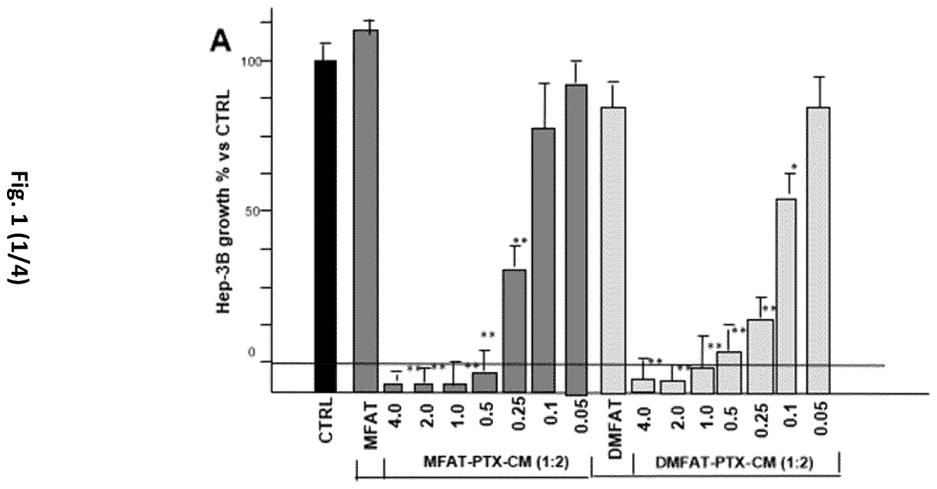

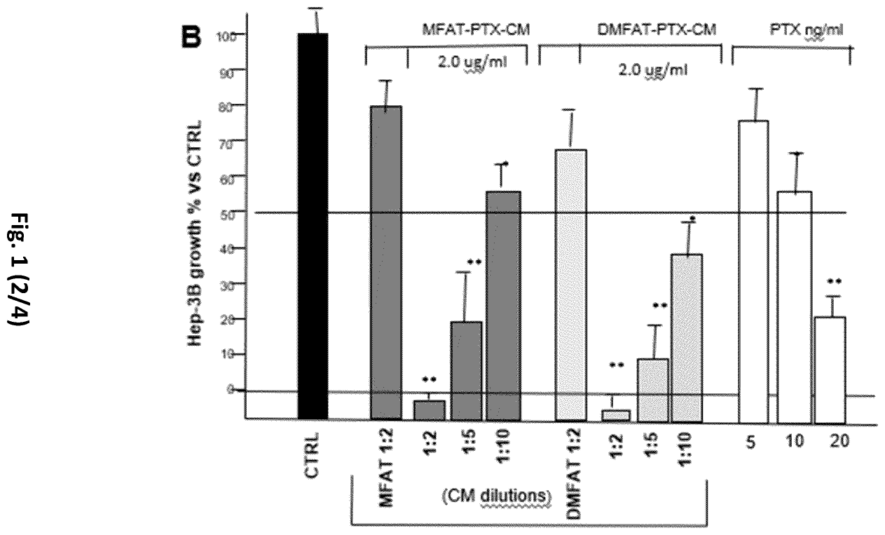

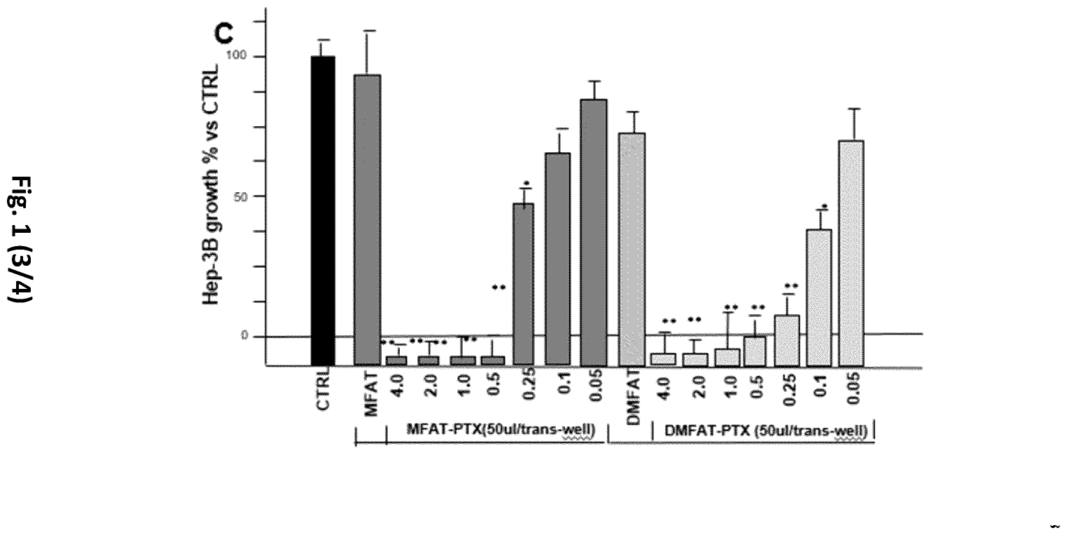

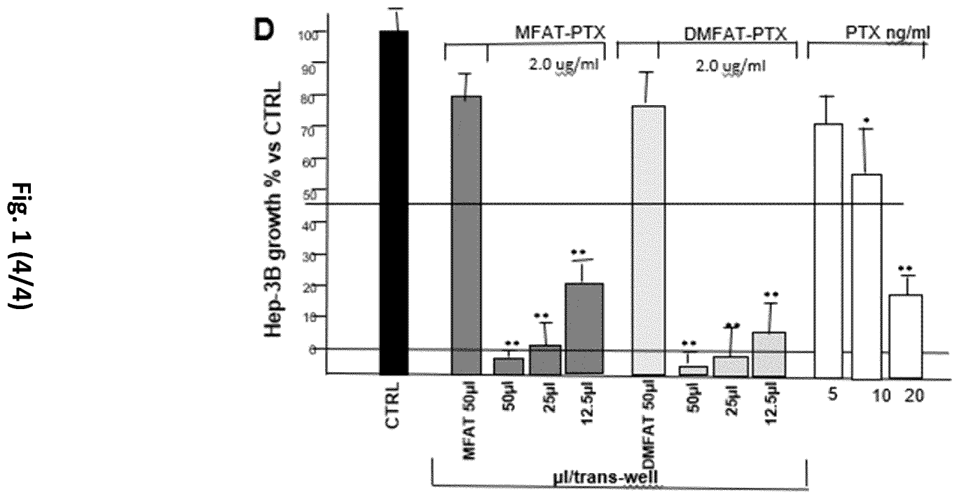

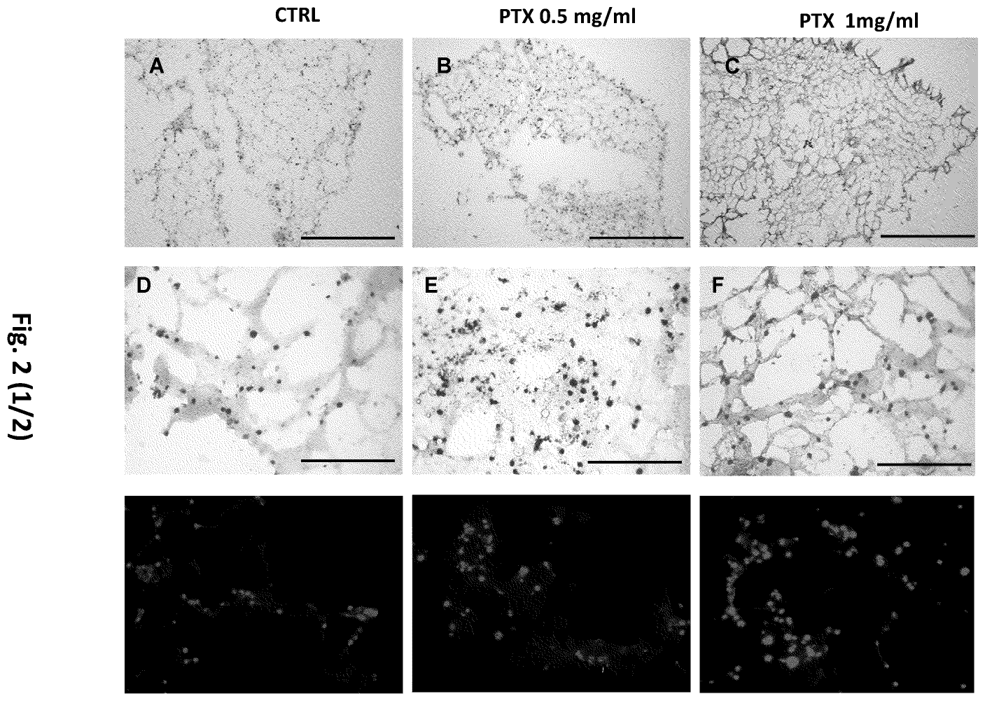

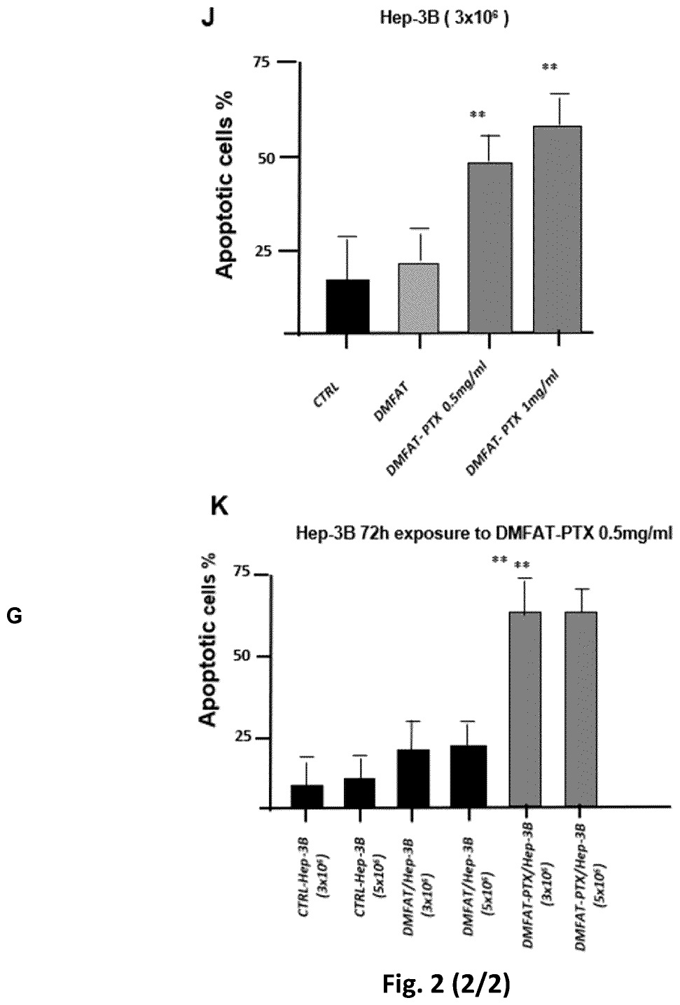

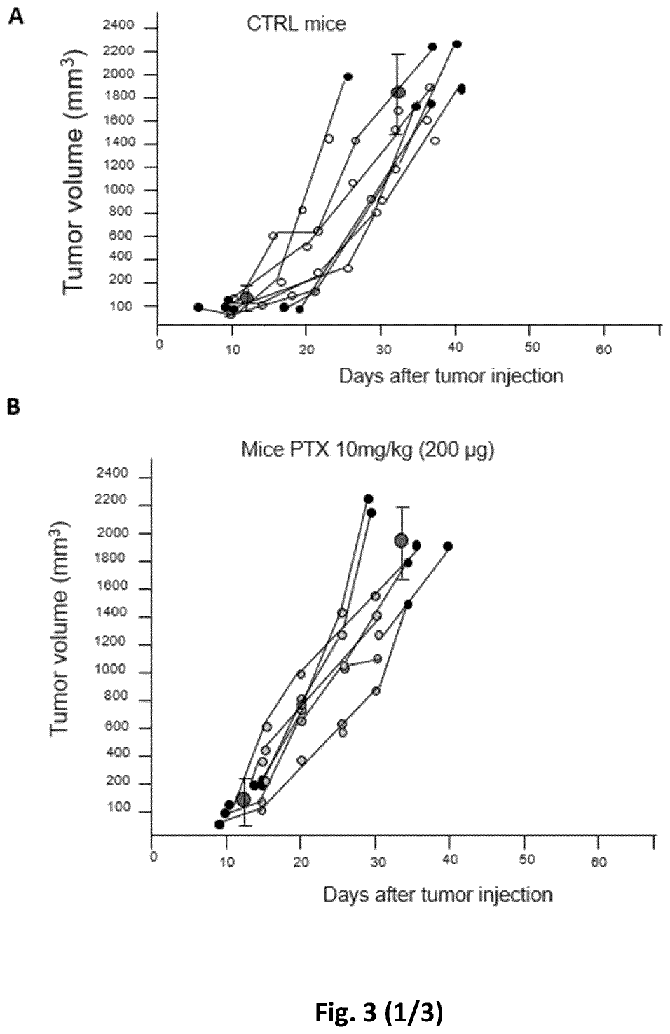

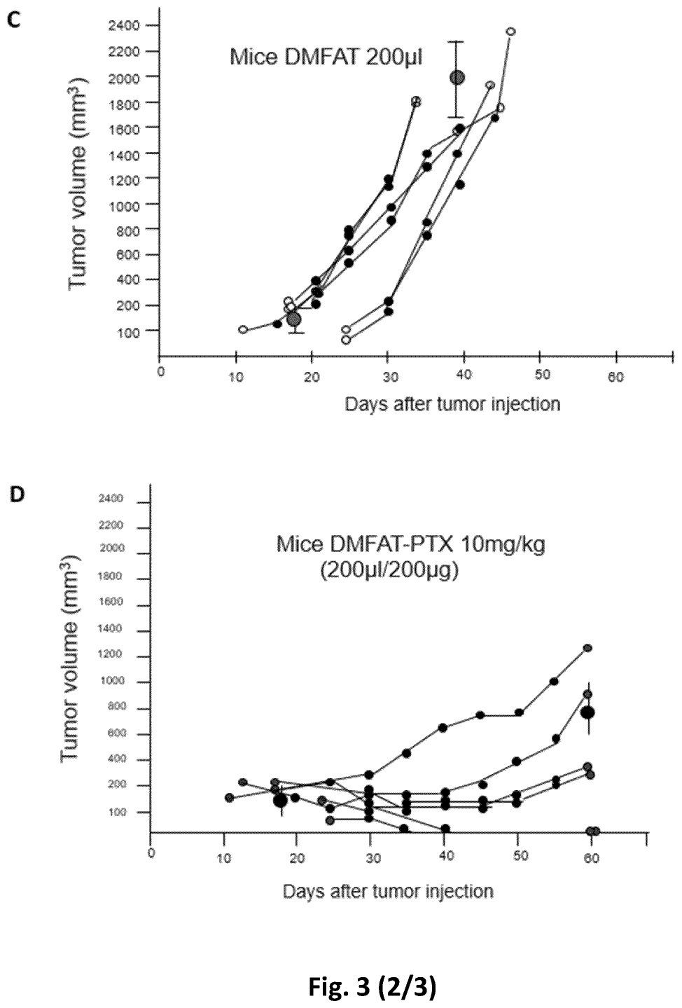

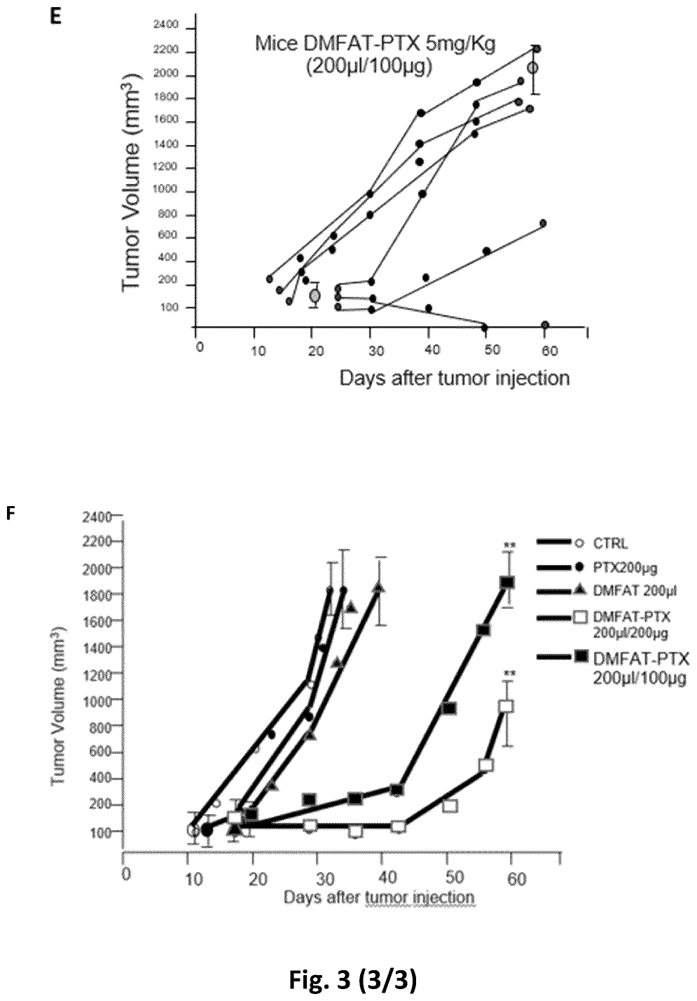

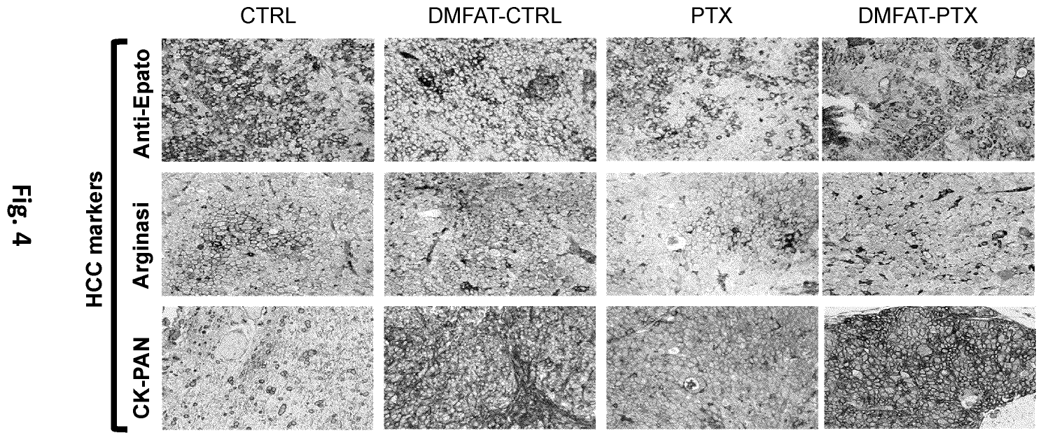

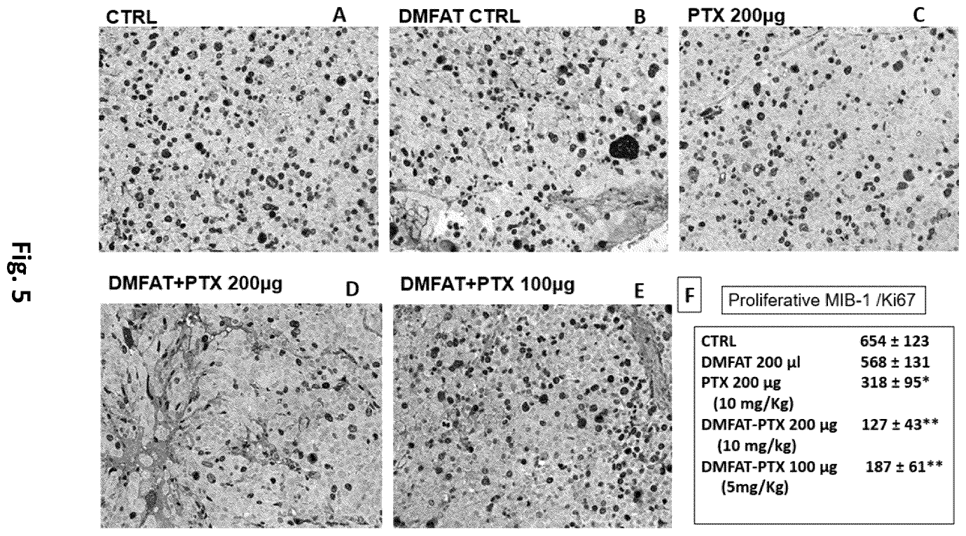

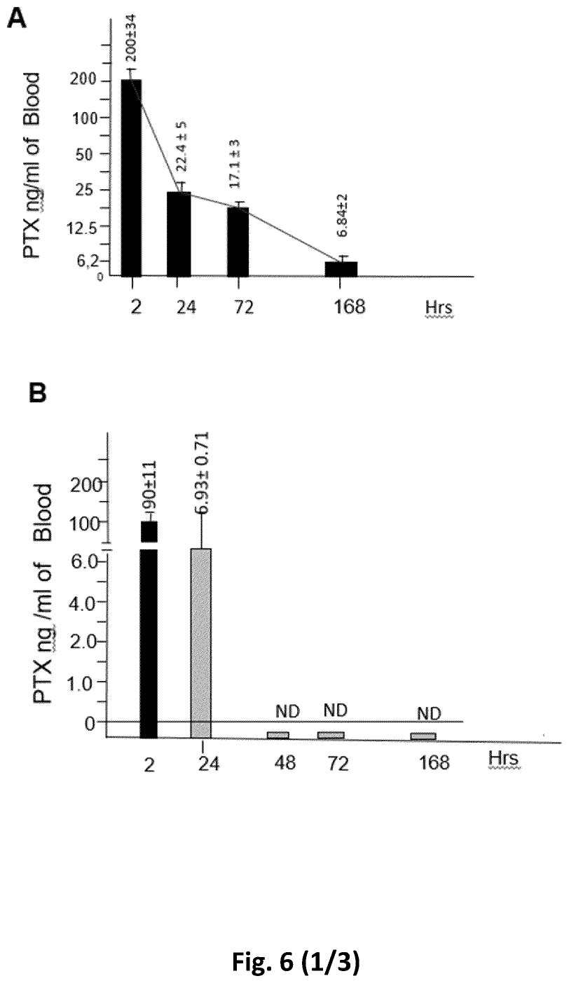

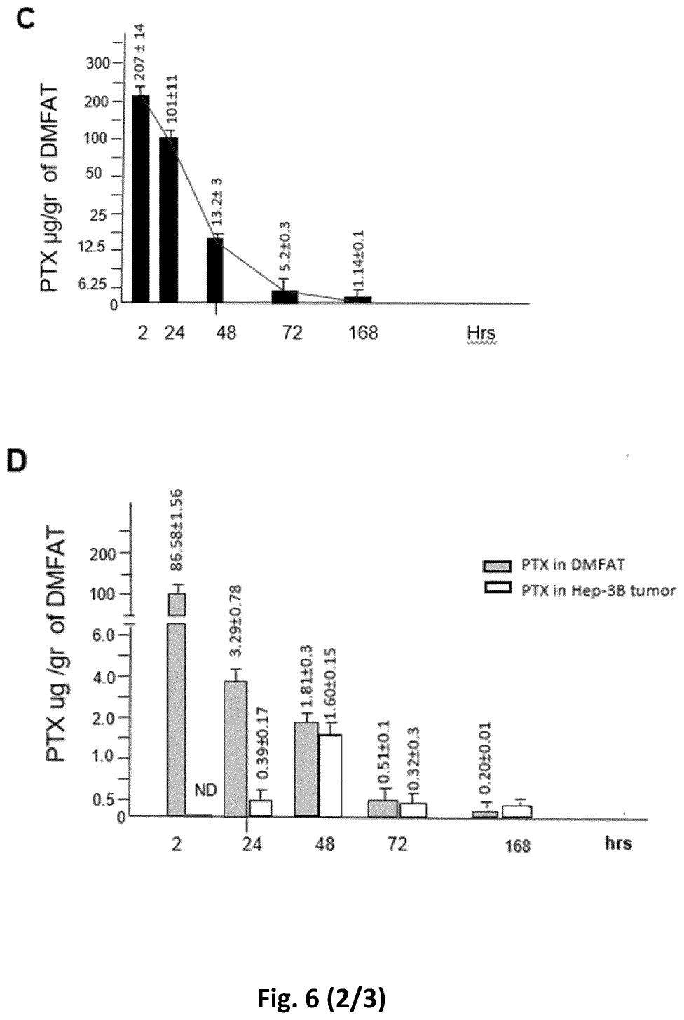

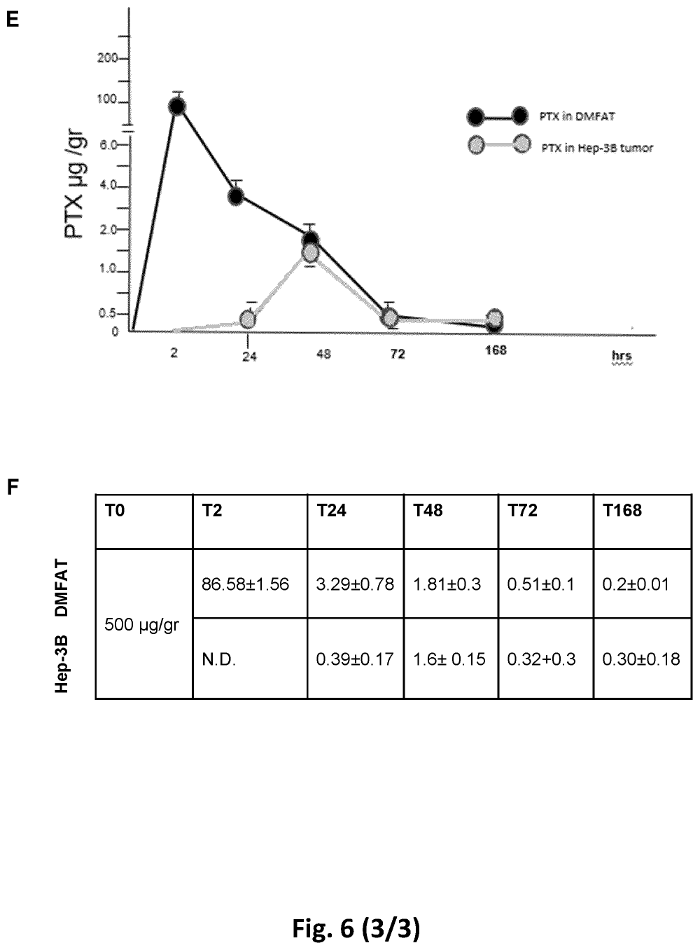

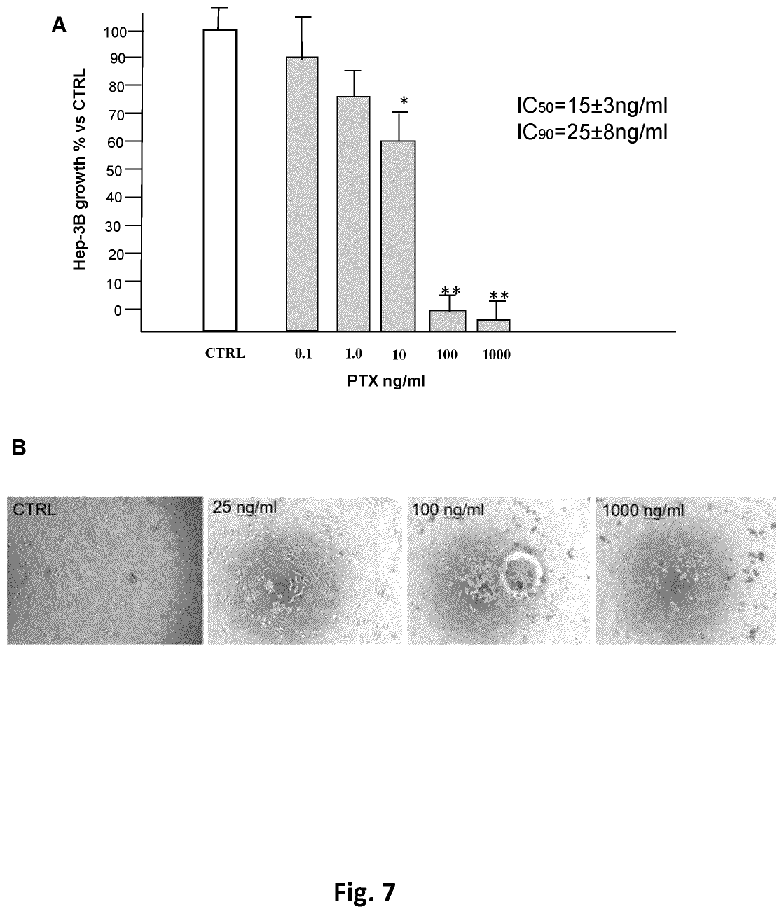

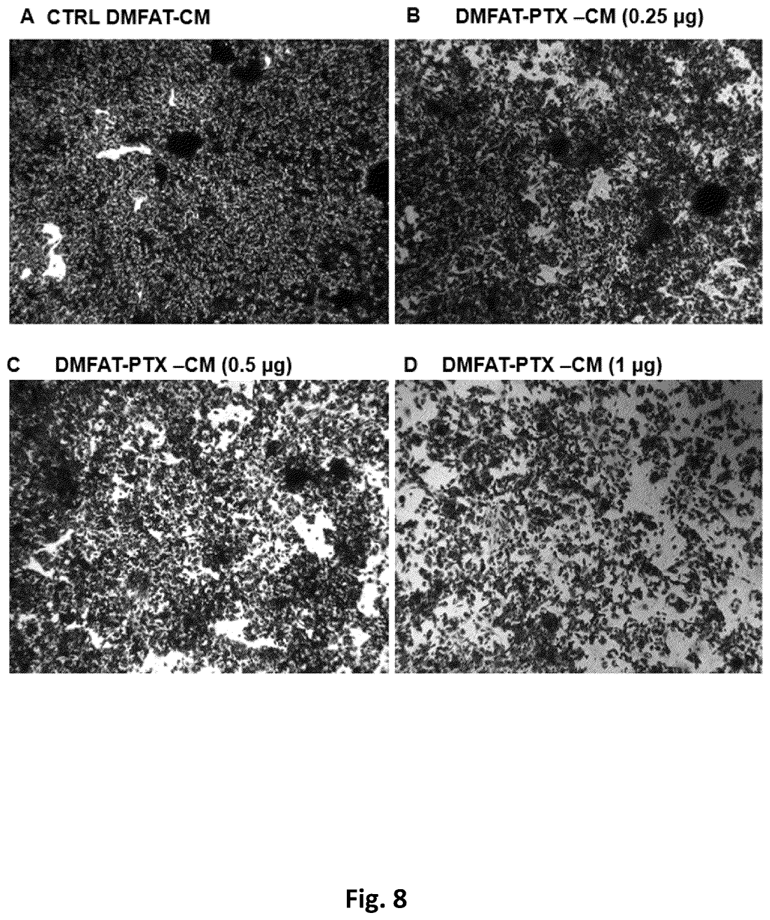

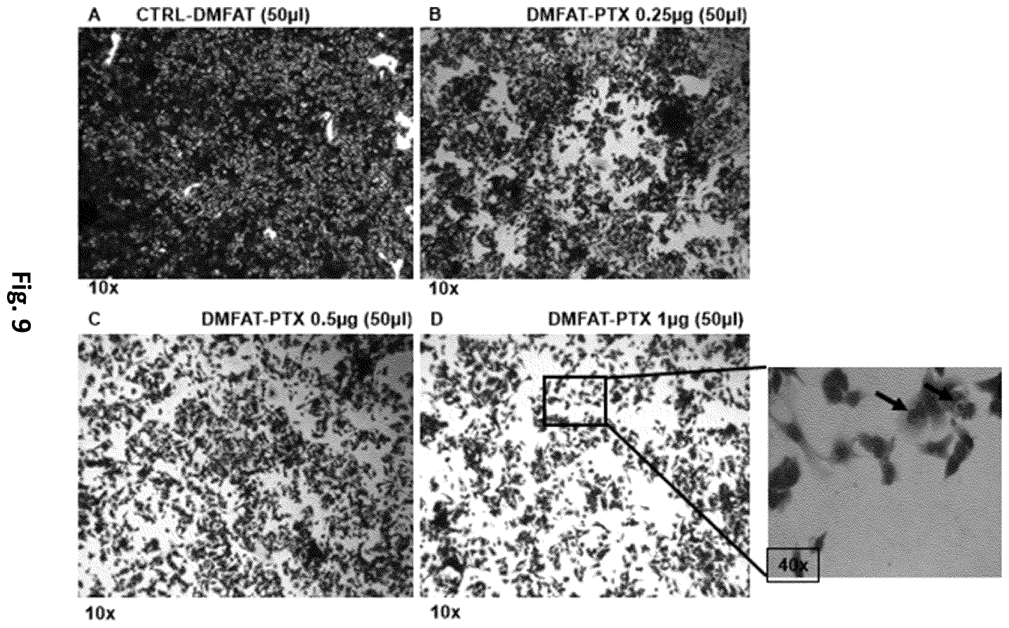

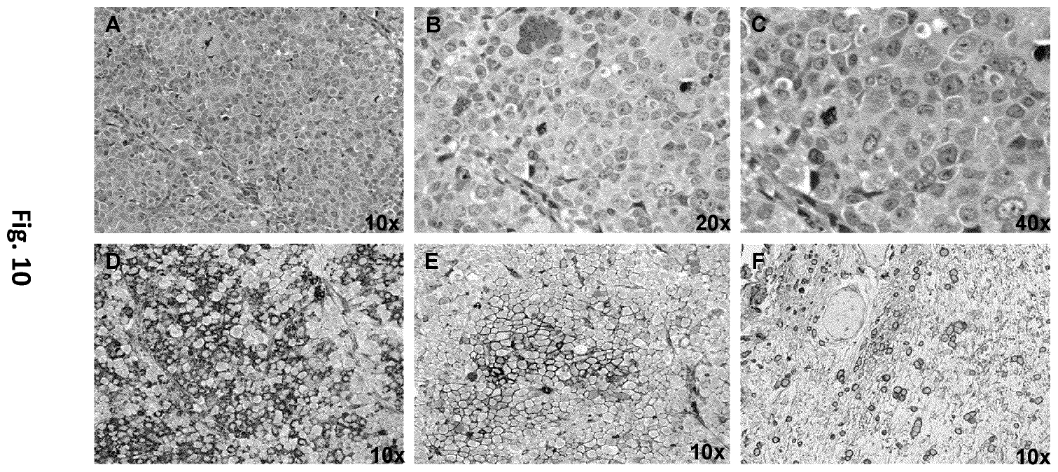

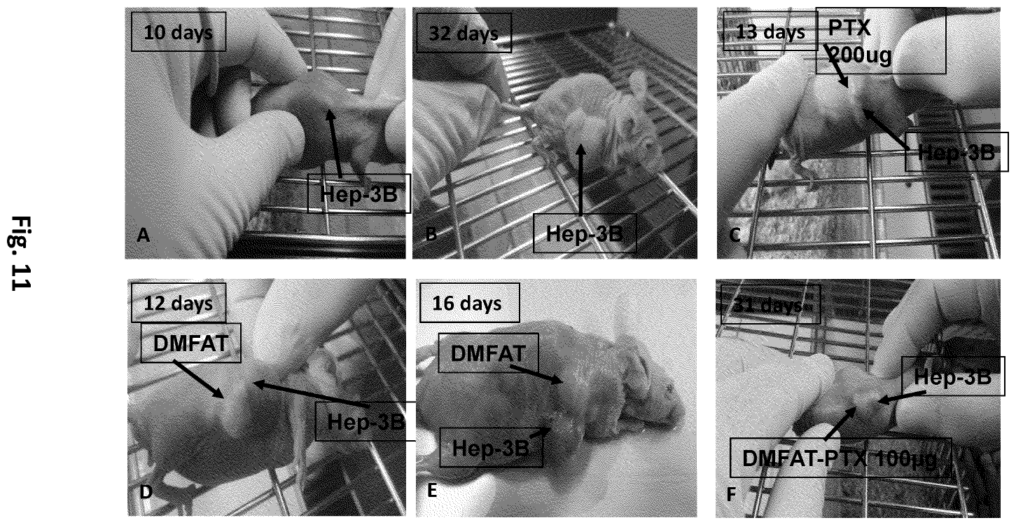

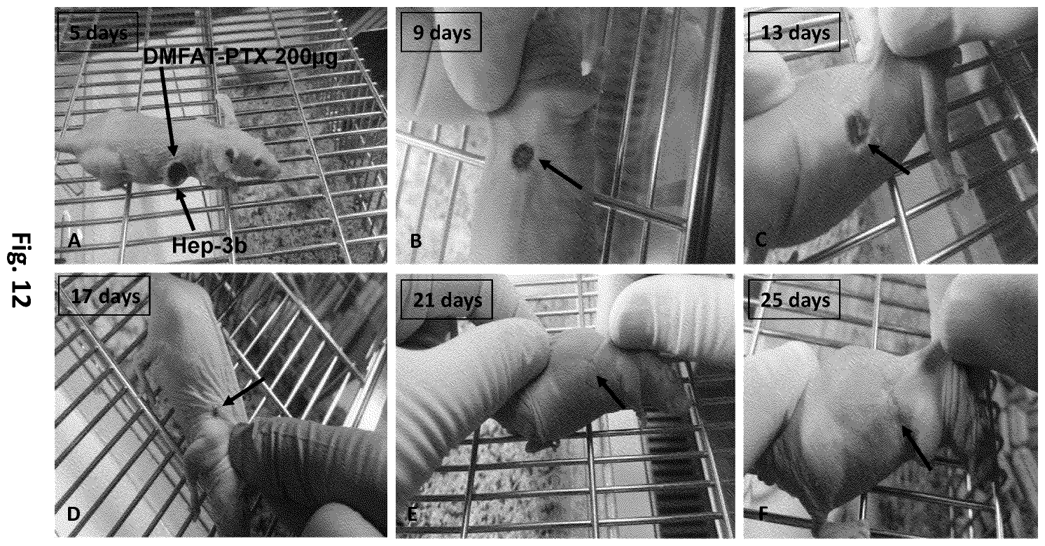

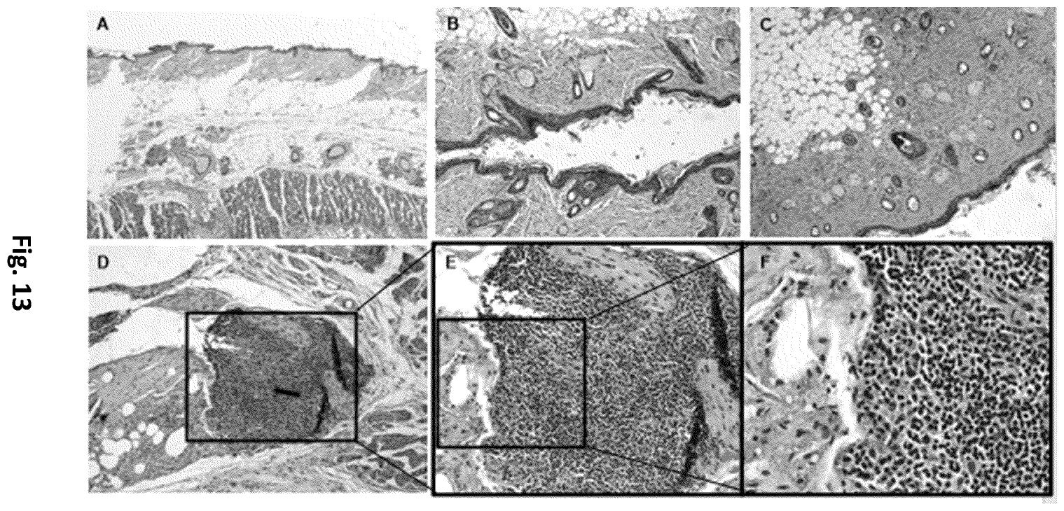

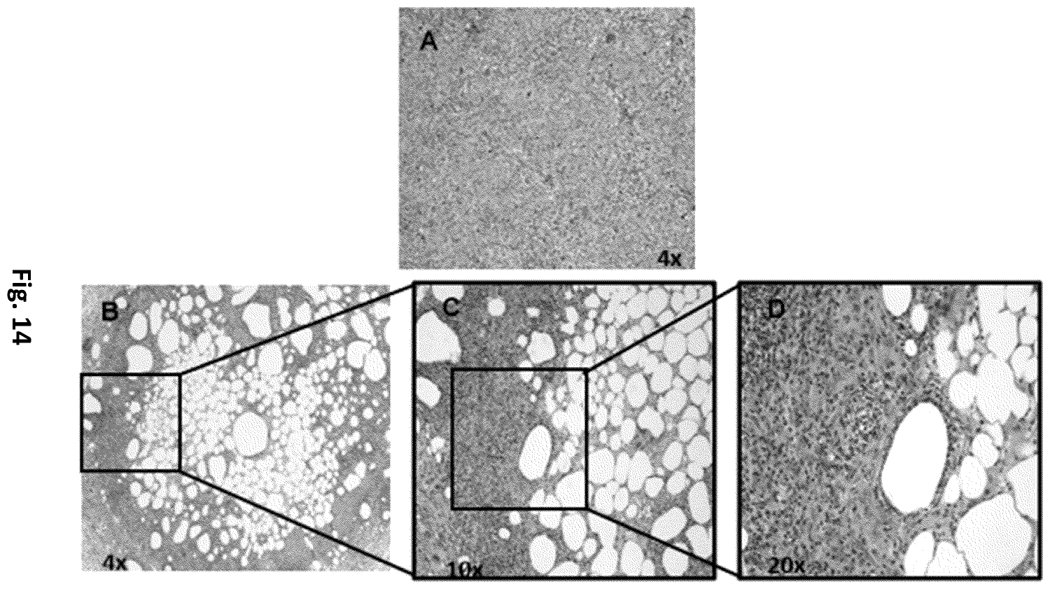

In the figure Hep-3B cells proliferation activity (expressed as % of control) in the presence of either conditioned medium (CM) (A and B) or co-cultured with fat specimens by trans-wells insert (C and D) is reported. FIG. 1A reports the anti-proliferative activity of CM (24 hours culture) from MFAT and DMFAT loaded with different amount of PTX (4 to 0.05 g/ml). FIG. 1B shows CM of DMFAT and MFAT loaded or not with 2 μg/ml PTX and tested at different dilutions. Experiments were run in parallel with free PTX addition to establish PTX equivalent concentration (p-EC). FIG. 1C reports results of trans-wells co-culture of MFAT and DMFAT specimens (50 μl) loaded with different amount of PTX (4 to 0.05 μg/ml), while FIG. 1D the anti-proliferative activity of different dose of MFAT and DMFAT specimens loaded with 2 μg/ml PTX, run in parallel, with free PTX addition. To note, CM and specimens recovered from DMFAT primed with PTX at 0.1 μg/ml were more effective than MFAT.

Columns in the figures are the means±SD of two separate experiments, done in triplicate. t-test: * indicate p<0.05; ** p<0.01 versus CTRL MFAT and DMFAT derived CM and specimens, respectively.

FIG. 2. Histological analysis of the 3D constructs mixing Hep-3B cells with control and DMFAT loaded with PTX

Hematoxylin and eosin (H&E) staining shows DMFAT with a typical adipose tissue structure (A-C). DMFAT appeared looser and more disaggregated in control (A) and loaded with PTX 0.5 mg/ml groups (B) while remaining more compact when loaded with PTX 1 mg/ml (C). Hep-3B cells were homogeneously distributed in cluster on DMFAT acting as a matrix scaffold and was more visible at higher magnification (D-F). The apoptotic effect of DMFAT loaded with PTX on Hep-3B cells detected with Hoechst 33342 staining is shown in G-I ((G) Control group, (H) 0.5 mg/ml and (I) PTX 1 mg/ml-treated group). Chromatin condensation, nuclear fragmentation and apoptotic bodies are indicated by arrows. Quantification of cell apoptosis was evaluated by Annexin V expression and presented as % of positive cells vs total cells recovered in the constructs incubated with DMFAT uploaded with different PTX concentrations (J) or in the presence of different amount of Hep-3B (K). Scale bars=400 μm (A-C), 200 μm (D-F). G-I scale bar of 200 mm. Columns in the figures are the means±SD of two separate experiments, done in triplicate. t-test: ** indicates p<0.01 versus CTRL DMFAT.

FIG. 3. Single-shot local injection of DMFAT-PTX inhibited Hep-3b growth in vivo.

Around 5×10⁶ Hep-3B were injected sc into nude mice. After 10-12 days, when the tumor was palpable (0.5-0.7 cm diameter around 100-200 mg in weight) mice were injected at the tumor site with saline 200 μl (A), PTX 200 μg/200 μl (10 mg/kg) (B), control DMFAT 200 μl (C), DMFAT-PTX 200 ul/200 ug (D) and DMFAT-PTX 200 ul/100 ug (E), respectively. Tumor volume was calculated by measuring the tumor diameters taken every two days with a calibre using the formula 1/6πd³. In the graphs the growth of tumor for each individual animal is shown. In (F) the average tumor growth of control and treated groups is shown. To note, that the single-shot injection of DMFAT-PTX at high dose (200 μg/200 μl) produced a significant delay of tumor growth; at 60 days 33% of the mice were tumor free (D). Reducing PTX concentration in DMFAT to 100 μg/200 μl still produced significant tumor growth inhibition (F). At the time of sacrifice, tumors were weighted; **p<0.01 vs saline control or vs DMFAT and free PTX treated mice.

FIG. 4. Immunohistochemical staining for typical biomarkers of HCC.

DMFAT-CTRL and PTX samples exhibited marked, diffuse positive reactivity for anti-Epato, arginase and ck-pan markers, similarly to CTRL group. In DMFAT-PTX, at both dosages used (images shown in figure refer to 100 μg/200 μl dosage), it was found lesser positivity for anti-Epato and Arginase and an increase in CK-Pan expression. Photographs obtained at 10× magnification.

FIG. 5. DMFAT-PTX treatment inhibited proliferative marker expression MIB-1/Ki67 in Hep-3B tumor.

The nuclear staining of the proliferation marker Ki67 is shown in CTRL group (A), in mice treated with DMFAT (B), PTX 10 mg/kg (C), DMFAT-PTX 10 mg/Kg (D) and DMFAT-PTX 5 mg/kg (E). A greater proportion positivity for Ki67 expression in CTRL and DMFAT groups was observed. In DMFAT-PTX treated groups this proportion was strongly reduced, particularly in DMFAT-PTX 10 mg/Kg. Some significant inhibition was observed also in free PTX treated group. FIG. 5F reports the quantification of the positive Ki67 cells for each group.

Numbers represent positive cells counted and represent means±SD of ten different fields (20× magnification). t-test: * indicate p<0.05; ** p<0.01 versus CTRL

FIG. 6. PK of PTX released by DMFAT-PTX in normal and in tumor bearing mice.

A and B show the blood concentrations of PTX released by DMFAT-PTX (5 mg/kg) given sc in normal and tumor bearing mice, detected at 2, 24, 72 and 168 hours after injection, respectively. As expected, the plasmatic concentration of PTX after sc treatment decreased rapidly but was detectable until 7 days in normal mice (A), whereas in tumor bearing mice PTX was detectable only until 24 hours suggesting a faster removal of the drug from the bloodstream (B). FIG. 6C shows the residual amount of drug values in the site of sc injection in normal mice. To note, at 2 hours the residual amount of PTX found in situ was about 40% of the one injected, whereas after 168 hours a significant amount of drug in the residual DMFAT specimens was still detected (up to 1 μg/g) and this must be considered of pharmacological importance. In (D) the PTX concentration in both residual DMFAT and in tumor specimens are shown. After 2 hours in situ PTX was only 17% of the quantity injected while in tumor was undetectable. PTX detection in tumor initiated at 24 hours, reaching a concentration equal to adjacent DMFAT at 48 hours, and remaining similar until 168 hours. At this time, the drug present in the residual DMFAT and in tumor specimens (0.2 μg/g in DMFAT and 0.3 ug/g in tumor) were, however, lower than those recovered in normal mice but were still higher than the IC₅₀ of the PTX. In (E) are shown the kinetics of PTX detected in both DMFAT and tumor specimens. In (F) are summarized the values of PTX concentration (expressed as μg/g of tissue) at different time upon DMFAT transplantation.

Numbers in the figures are the means±(up to 1 μg/g) SD.

FIG. 7. PTX displayed anti-proliferative activity on Hep-3B cells in culture.

In (A) PTX at different concentration from 0.1 to 1000 ng/ml was added to cultured Hep-3B cells After 72 hours of incubation cells were detached and counted. PTX showed an IC50 at 15±3 ng/ml and IC90 at 25±8 ng/ml. In (B) photographs (10× magnification) of Hep-3B cultured in presence of PTX showing its potent growth inhibition at concentration up to 25 ng/ml are reported.

Columns in the figure are the % of growth versus CTRL medium and are the Means±SD of three separate experiments, performed in triplicate. t-test: * and ** indicate p<0.05 and p<0.01 respectively.

FIG. 8. Effect of DMFAT-PTX-CM on Hep-3B growth.

Representative images of Hep-3B cancer cell cultures stained with 0.25% crystal violet upon 72 hours of treatment with CM (1:2 dilution) from control DMFAT (A), DMFAT-PTX (0.25μ/ml) (B), DMFAT-PTX (0.5 μg/ml) (C), DMFAT-PTX (1 μg/ml) (D), respectively. Photographs (10× magnification) show the potent anti-cancer activity of CM from DMFAT-PTX.

FIG. 9. Effect of DMFAT-PTX specimens (50 μl) placed in the upper well of a trans-well insert.

Hep-3B seeded in the lower well of a trans-well insert and stained with 0.25% crystal violet solution upon 72 hours of incubation are presented. Photographs (10× magnification) show the effect of control DMFAT (A), DMFAT-PTX (0.25μ/ml) (B), DMFAT-PTX (0.5 μg/ml) (C) and DMFAT-PTX (1 μg/ml) (D) specimens on Hep-3B, respectively. In the high magnification box (40×) the presence of cells with apoptotic features are indicated by block arrows.

FIG. 10. Immunohistochemical characterization of Hep-3B-induced HCC in mice. H&E staining revealed a vascularized tumor with polygonal cells (A), central vacuolar nuclei and prominent nucleoli with some pleomorphic and multinucleated giant cells, more easily visible at higher magnifications (B and C). Immunohistochemical staining confirmed the hepatic origin of the tumor (10× magnification). Cells showed distinct granular cytoplasmic staining for anti-Epato (D), and moderate to strong, diffuse reactivity for Arginase-1 (E). Pan-CK marker was present on the vast majority of Hep-3B cells and showed a distinct cytoplasmic staining reaction with membrane accentuation (F).

FIG. 11. Representative photographs of mice injected in the right flank with Hep-3B cells and treated with DMFAT loaded or not with PTX

In (A) the control tumor nodule at 10 days upon Hep-3B injection is shown while in (B) the control mice treated with saline with large tumor (>2 cm in diameter) at 32 days (just before sacrifice) is presented. Panel (C) shows the tumor at day 12, injected locally with free PTX. (D) and (E) show two different mice with DMFAT (200 ul) placed just beside the tumor nodule, while in (F) a case with the tumor treated with DMFAT-PTX (5 mg/kg) at day 31 is presented. To note the relative small dimension of the tumor and the absence of skin ulceration.

FIG. 12. Local injection of DMFAT-PTX (200 μg/200 ul) induced Hep-3B regression but also skin lesion that healed spontaneously.

Photographs of mice, injected in the right flank with Hep-3B cells and then treated locally with DMFAT-PTX (10 mg/kg) that were taken at various time intervals: 5 days (A), 9 days (B), 13 days (C), 17 days (D), 21 days (E) and 25 days (F) after treatment, respectively. To note, all lesions healed spontaneously without the need of any particular pharmacological treatment in around 2-3 weeks. Block arrows indicate the area of tumor and DMFAT-PTX injections.

FIG. 13. Skin histology of mice (90 days) affected by the HCC tumor when cured.

H&E staining of skin sections showed no observable damage to mice skin after wound healing in specimens treated with DMFAT-PTX 200. Epidermal layer with stratum corneum (A), dermis housing glands and blood vessels (B), hypodermis containing adipose cells and skeletal muscle are visible (C). In (D) images showing a stromal region-infiltrating by lymphocytes at 4× magnification are presented; at higher magnifications (10× (E) and 20× (F)) foreign body cell (arrowhead) and reactive lymphoid cell (black arrow) are easily noticed.

FIG. 14. Histopathological analysis of the HCC tumor induced by Hep-3b cells treated with DMFAT.

In (A) necrotic areas characterized by foci of cells with eosinophilic cytoplasm and pyknotic or karyorrhexic nuclei in the HCC are reported. Tumor mass treated with DMFAT was characterized by a strong steatosis due to a wide distribution of lipid drops (B). At higher magnifications, steatonecrosis areas with infiltration of inflammatory cells, predominantly neutrophils around fat vacuoles, could be detected (C, D).

### MATERIAL AND METHODS

### Sample Collection, Ethics Statements, MFAT and DMFAT Preparation

Samples of Lipoaspirate (LP) were obtained by liposuction of subcutaneous tissue as previously described elsewhere by using disposable cannulas provided with the Lipogems® kit (7, 8). Tissue samples were collected from plastic surgery operations after signed informed consent by the patient, in accordance with the Declaration of Helsinki. The approval for their use was obtained from the Institutional Ethical Committee of Milan University (n.59/15, C.E. UNIMI, 09.1115). For all the in vitro and in vivo experiments performed in this study fat tissue was obtained from five different human donors that underwent plastic surgery. MFAT specimens were obtained as previously described (7, 9). Briefly, by using a standard 225-ml Lipogems® device (provided by Lipogems® International, Milan, Italy), LP collected by syringe was pushed into the Lipogems® device through a filter for a first clusters reduction and complete disaggregation was obtained by shaking the device containing inside five stainless steel marbles. Afterwards, the micro fragmented fat tissue was aspirated by a syringe connected with the device and was ready for experiments. Devitalized MFAT (DMFAT) was prepared following a previously published procedure (6,10) consisting of 3 freeze (−20° C.) and thaw (F/T) cycles. Briefly, aliquots of MFAT (5 ml) were transferred in a conical tube washed with PBS three times by centrifugation at 200 g×10′. After discarding PBS washing solution, MFAT undergoing a freeze (−20° C.) and thaw (F/T) cycles (usually 3 of 30′ each) that lead to the killing of all the cells in the stromal vascular fraction (SVF). Aliquots of DMFAT (1-2 ml) were kept at −80° C. until use for experiments, others were analyzed to verify the absence of cell vitality by SVF extraction with collagenase (SIGMA St. Louis, Mo, USA) (0.2% w/v) following processing the final cell pellets by Tripan-blue assay. To note that DMFAT specimens before used for in vitro and in vivo experiments were washed several times with PBS by centrifugation in order to remove as much as possible all cell debris and the residual presence of membrane proteins and genetic material content and then investigated as previously described (10).

### Chemicals and Reagents

Methanol, isopropanol, acetonitrile and formic acid (all analytical grade) were supplied from Merck (Darmstadt, Germany). Ammonium formate was purchased from Sigma Aldrich (St. Louis, MO, USA) Water was MilliQ-grade. Paclitaxel (PTX) and PTX-D5 were purchased from Cabru (Arcore, Italy). To treat the fat samples a clinical preparation of PTX (stock solution of 6 mg/ml, Fresenius Kabi, Italy) was used.

### Tumor Cell Lines

The in vitro and in vivo anti-cancer activity of PTX loaded into MFAT and DMFAT was evaluated on Hep-3B, an HCC cell line. This cell line was kindly provided by Dr. Valentina Fonsato (Molecular Biotechnology Center (MBC), University of Turin, Turin Italy) and was purchased from ATCC (ATCC® HB-8064™). Hep-3B was maintained following ATCC instructions. Briefly, cells were cultured in Eagle's Minimum Essential Medium (MEM) (Euroclone, UK) supplemented with 10% fetal calf serum FCS (Gibco, Life Technologies, Monza, Italy) and passed weekly at ratio 1:5.

### Procedure for PTX Priming of MFAT and DMFAT Specimens for In Vitro Experiments

MFAT and DMFAT specimens were primed with PTX following published procedure (6). Briefly, upon Phosphate Buffered Saline (PBS) washing by centrifugation (200 g×10 minutes) around 1 ml of MFAT and DMFAT specimens were mixed with different concentration of PTX (ranging from 0.05 to 4 μg/ml) and prepared fresh from a stock solution 6 mg/ml and diluted in MEM+0.2% Bovine serum albumin (BSA). Then, samples were shaken and incubated for about 30 minutes (at 37° C., 5% CO₂). At the end of incubation MFAT-PTX and DMFAT-PTX specimens were washed twice (200 g×10 minutes) with PBS to remove unbound PTX. Control untreated MFAT and DMFAT specimens were similarly processed. At this point, both primed and un-primed MFAT and DMFAT specimens were considered ready for studying their biological activity in vitro.

Evaluation of the activity of MFAT and DMFAT loaded with PTX on Hep-3B growth in 2D assay

MFAT-PTX and DMFAT-PTX preparations were evaluated for anti-cancer activity either by using trans-well inserts in a co-culture assay with Hep-3B cells, or by evaluating the anti-proliferative activity of their conditioned medium (CM) obtained by incubating 24 hours (37° C., 5% CO₂) around 1 ml of MFAT-PTX and DMFAT-PTX specimens (loaded with various PTX concentrations 0.05-4 ug/ml) cultured in similar volume of MEM complete medium. The direct anti-cancer activity of MFAT-PTX or DMFAT-PTX specimens was tested by using trans-well inserts (0.4 μm pore size; BD Falcon, NJ, USA). Briefly, around 2×10⁴ Hep-3B cells were seeded in wells (24-multiwell plate) and then covered with 700 l/well of complete MEM medium and left to adhere 3 hours. Next, different volumes (50, 25, and 12.5 μl) of MFAT-PTX, DMFAT-PTX or control untreated specimens were placed in the upper compartment of trans-well inserts, covered with 200 μl of medium and then placed in the wells with cancer cells. After 72 h of incubation, the direct effect of fat specimens on the tumor cell growth was evaluated either by detaching and counting the Hep-3B in the wells or, in a more limited series of experiments, by staining the adherent Hep-3B cells with 0.25% crystal violet (Sigma Aldrich, USA) and evaluating the optical density of the eluted dye obtained by cell lysing (6).

The anti-cancer activity of CM derived from both MFAT-PTX or DMFAT-PTX specimens was evaluated in a 72-hours proliferation assay as previously described (6, 11). Briefly, around 2×10⁴ Hep-3B cells were seeded in wells (24-multiwell plate) and then covered with 500 l/well of complete MEM medium and left to adhere 3 h. Then CM, at different dilutions (from 1:2 to 1:10), derived from cultured MFAT-PTX, DMFAT-PTX and from control untreated specimens, were added to wells and further incubated for 72 hours. At the end of incubation cancer cells were detached with trypsin and counted as previously described (11). The anti-tumor activity of CM from MFAT-PTX and DMFAT-PTX were compared to the one of pure PTX and expressed as PTX equivalent concentration (p-EC) according to the following algorithm p-EC (ng/ml)=IC₅₀ PTX×100/V₅₀ (μl/well) where IC₅₀ PTX is the concentration of pure PTX producing 50% growth inhibition and V₅₀ the respective volume of CM that produces the same inhibition.

The anti-tumor activity of CM from MFAT-PTX and DMFAT-PTX were compared to the one of pure PTX and expressed as PTX equivalent concentration (p-EC) according to the following algorithm p-EC (ng/ml)=IC₅₀ PTX×100/V₅₀ (μl/well) where IC₅₀ PTX is the concentration of pure PTX producing 50% growth inhibition and V₅₀ the respective volume of CM that produces the same inhibition.

### Histological Analyses of DMFAT—Hep-3B Cells in 3D Constructs

The efficacy of MFAT-PTX or DMFAT-PTX specimens on Hep-3B was also investigated in a 3D assay. Briefly, 50 μl of control or PTX loaded MFAT specimens (at 0.5 at 1 mg/ml) were mixed at 4° C. with 100 μl of Matrigel (BD Biosciences, Franklin Lakes, NJ, USA) where Hep-3B (3 and 5×10⁶) cells were added and left to jellify for one hour at 37° C. Then, complete growth MEM was added to gels and further incubated for 72 hours. At the end, medium was removed, and gel processed by immunocytochemical analysis through cyto-inclusion technique (12). Samples were fixed in 4% paraformaldehyde (PFA) and cryoprotected overnight at 4° C. by immersion in a 30% (wt/vol) sucrose solution before being embedded in Tissue-Tek O.C.T. Compound (Tissue-Tek; Sakura Finetek, Tor-rance, CA, www.sakuraus.com.) and frozen. Sections were cut 5-μm thick with a cryostat at −20° C. and stained with hematoxylin and eosin (H&E) (Sigma-Aldrich, St. Louis, MO, USA) or Hoechst 33342 (Thermo Fisher Scientific) to detect apoptotic cells. Samples were visualized using conventional light or fluorescent microscopes. In other series of experiments apoptosis was investigated by using Annexin V staining (Thermo Fisher Scientific). Briefly, after 72 h incubation, Hep-3B cells were extracted from 3D constructs by digestion with collagenase (Sigma). After cells washing by centrifugation, Fluorescent Annexin V conjugates was used in flow cytometry (FC) as previously described (6).

### Histology of Hep-3B Tumor Specimens In Vivo

All tumor specimens were formalin 10% embedded, included in paraffin; sections with Hematoxylin and Eosin (H&E) staining were performed. After evaluation of conventional H&E stained, ten white sections were then obtained from each sample to be processed for immunohistochemical (IHC) staining. IHC was carried out automatically by means of Leica Bond Max™ technology (Arginase, CK-PAN, CK7 and ANTI-EPATO) and Ventana Bench Mark Ultra (Ki-67): ANTI-HEPATO: antigen retrieval 30′ EDTA, (dil. 1:300 clone OCHIES Dako-Agilent); ARGINASI antigen retrieval 30′ EDTA, (dil. 1:100 Clone SP156 Cell Marque Diapath) KI-67: T.Q. clone 30-9 Roche-Ventana); CK-PAN: antigen retrieval 5′ Enzyma, (dil. 1:200 clone MNF116 Dako Agilent); CK-7: antigen retrieval 15′ EDTA, dil. 1:100 clone OV-TL 12/20 Dako Agilent. All section stained was evaluated on NIKON ECLIPSE E 600 Microscopy equipped with OLYMPUS DP21 Camera.

### Evaluation of In Vivo Anti-Tumor Activity of DMFAT-PTX

Five-week-old athymic nude-Foxn1nu mice were purchased from Envigo (Envigo, Bresso, Italy) and were housed under pathogen-free conditions. Experiments were reviewed and approved by the licensing and ethical committee of IZSLER (Istituto Zooprofilattico Sperimentale della Lombardia e dell'Emilia Romagna, Brescia, Italy) and by the Italian Ministry of Health. To note, for these experiments only DMFAT specimens were used, because of the advantage given by the possibility to conserve frozen biomaterials until use in mice.

In a preliminary experiment, 3 mice were injected subcutaneously (s.c.) with 5×10⁶ Hep-3B cells in 100 ul of MTG in the right flank. Mice were observed daily to establish the day of tumor appearance and when the nodule was 1 cm in diameter, mice were sacrificed and tumor was removed and investigated to verify the HCC features by histological examination. To note, for these experiments only DMFAT specimens were used, because of the advantage given by the possibility to conserve frozen biomaterials until use in mice. In the first series of experiments, mice (n=6/group) were injected with 5×10⁶ Hep-3B cells in the right flank (day 0). The tumors were allowed to grow to an average 0.5/0.7 cm in diameter corresponding to a tumor volume ranging from 65 to 179 mm³ (median weight 120 mg) that were calculated using the formula 1/6πd³ (13). After 10-14 days the mice were randomly subdivided into 4 groups and treated just next to the tumor nodule with a single shot of 200 μl saline (control group CTRL), DMFAT (200 μl), DMFAT-PTX 10 mg/kg (200 μg/200 l) and free PTX drug (200 μg/200 μl saline), respectively. Loading of PTX into just thawed DMFAT specimens were performed 20-30 minutes before injection as described above, adding 1 mg of PTX (around 166 μl of the stock PTX solution 6 mg/ml) to 1 ml of DMFAT and agitation. After treatments, mice were observed daily; every two days tumor diameters were measured by caliber. According to the ethical protocol, all the mice were sacrificed when the tumor nodule reached 2.0-2.5 cm in diameter (≥2 g of weight) or, whatever the diameter, sacrificed on day 60 after the transplant. At this time only tumor-free mice were followed until 90 days. Animals were euthanized with carbon dioxide inhalation, followed by cervical dislocation.

In a second series of experiments mice (n=6/group) were similarly injected s.c. with 5×10⁶ Hep-3B and treated locally with half dose of PTX, DMFAT-PTX (100 μg PTX/200 l) corresponding to 5 mg/kg.

Pharmacokinetics (PK) of PTX Released by DMFAT-PTX and Incorporated into Tumor Nodule

PK of PTX in cancer cells when DMFAT-PTX is located nearby was studied in another series of tests. To this end, mice (n=3 group) were injected subcutaneously (sc) with Hep-3B and when the tumor mass was palpable mice received 200 μl of DMFAT loaded with 100 μg PTX (dose 5 mg/kg) placed sideways to the tumor. Mice were then sacrificed at 2 hours, 1, 2, 3 and 7 days after treatments, blood, tumor nodule as well as residual subcutaneous DMFAT-PTX tissue were recovered, placed in a tube and rapidly stored (−80° C.) until use for evaluation of PTX content by mass spectrometry. Another group of tumor-free mice received a same sc injection of DMFAT-PTX (at the same dose 5 mg/kg PTX) to investigate the release of PTX in the absence of cancer nodule.

Biological samples, consisting in whole blood (100 μl), tumor and fat tissues homogenate (1-5 mg), were added with 25 μl of IS (Paclitaxel D5, PTX-D5 10 μg/ml) and extracted by single-step liquid extraction with methanol/isopropanol (60:40, v/v). Dry extracts were redissolved with 150 μl of acetonitrile/water (1:1, v/v), clarified on 45 μm filter and 5 μl injected for LC-MS/MS analysis. All samples were extracted twice. The LC-MS/MS consisted of a Shimadzu UPLC coupled with a Triple TOF 6600 Sciex (Concord, ON, CA) equipped with Turbo Spray IonDrive.

### Plasma Extraction Procedure

Extraction and purification from plasma was performed by SPE. 50 μL plasma was added with 100 μl of IS (PTX D5 0.1 μg/mL) and 850 μL of water, then sonicated for 30 min at 40° C. (Sonorex, Bandelin electronic, Berlin). Samples were centrifuged for 5 min at 10000 rpm (MiniSpin, Eppendorf, Hamburg). Solid-phase extraction was performed on Strata™-X 33 μm Polymeric Reversed Phase SPE 30 mg/l mL extraction cartridges from Phenomenex (Anzola Emilia, Italy) connected to Visiprep Solid Phase Extraction Vacuum Manifolds from Supelco (Bellefonte, USA). Before use, the cartridges were conditioned with 1 ml methanol and 1 ml deionized water. The diluted samples were percolated through the cartridges. The cartridges were then rinsed with 1 mL deionized water with 5% meOH, and vacuum-dried for 5 min to remove excess water. Finally, the retained compounds were eluted with 1 ml of methanol/isopropanol/formic acid (60:39.2:0.8) and the elution was collected in a test tube. The eluent was evaporated until dryness by a gentle nitrogen stream. Finally, the residue was re-dissolved with 150 μl acetonitrile and 10 μl were injected for LC-MS/MS analysis.

### Tissue Extraction Procedure

Extraction and purification from different tissues (s.c. injection area, and tumor sample) was performed by single-step extraction. Weighted tissues (10-50 mg) were homogenized in 100 μl of methanol by TissueLyser LT (Qiagen, Hilden, Germany) for 3′ at 50 oscillations/s. Samples were added with 100 μl of IS (Paclitaxel D5 0.1 μg/mL) and 800 μl of methanol/isopropanol/formic acid (60:39.2:0.8), then sonicated for 30 min at 40° C. The extract was evaporated until dryness by a gentle nitrogen stream. The tissues residue was re-dissolved with 150 μl of methanol, centrifuged for 10′ at 10,000 rpm, filtered through a NY 0.45 μm filter (LLG labware, Meckenheim) and transferred in a vial and 10 μl were injected for LC-MS/MS analysis.

### LC/MS-MS Conditions

The analytical system consisted of a IPLC coupled to a tandem mass spectrometer. The liquid chromatograph system was a Dionex 3000 UltiMate instrument with autosampler, bi-nary pump and column oven (Thermo Fisher Scientific, USA). Separation was attained on a reversed-phase Luna C18(2) 50 mm×2, 3 μm particle size (Phenomenex, California, USA) analytical column, preceded by a security guard cartridge with a linear gradient between eluent A (water+5 mM ammonium formate+0.1% formic acid) and eluent B (acetonitrile+0.1% formic acid). The column was equilibrated with 20% (B) for 2 min, increased to 95% (B) in 4 min, held for 0.5 min, back to the initial conditions in 0.5 min and kept for 2 min at 20% (B). The flow rate was 0.4 ml/min, the autosampler and the column oven were kept at 15° C. and 30° C., respectively. The tandem mass spectrometer was an AB Sciex 3200 QTRAP instrument with electrospray ionization TurboIonSpray™ source (AB Sciex S.r.l., Milano, Italy). Instruments were managed with the proprietary manufacturer's software and according to the manufacturer's instructions. The analytical data were processed using Analyst software (version 1.6.2). The ion spray voltage was set at 5.5 kV and the source temperature was set at 400° C. Nitrogen was used as a nebulizing gas (GS 1, 40 psi), turbo spray gas (GS 2, 45 psi) and curtain gas (30 psi). The collision-activated dissociation (CAD) was set to a medium level. The dwell time was set at 0.3 s, and the MS scan was performed in positive ion modes (ESI+). The product ion spectrum (MS-MS) was generated at optimized DPs to identify the prominent product ions of the analytes using nitrogen as the collision gas. The collision energies (CE) of product ions transition were optimized by CE ramping via direct infusion. Multiple reaction monitoring (MRM) mode was used. In table 1 are reported the optimal compound-dependent parameters.

TABLE 1
MS condition for each analyte, in bold
transition used for quantification

| Analytes | Transition | DP (eV) | EP (V) | CE (V) |
|---|---|---|---|---|
| Paclitaxel | 854.5 > 286.1 | 28 | 10 | 27 |
| | 854.5 > 509.0 | 28 | 10 | 17 |
| Paclitaxel D5 (IS) | 859.4 > 291.5 | 28 | 10 | 19 |

### Method Validation: Linearity and LOQ

The linearity was proven according to the regression line by the method of least squares and expressed by the coefficient of correlation (R²). Six-point matrix-matched calibration curves were evaluated by spiking increasing amounts of the analyte in blank plasma. Calibration curves were obtained by plotting the ratio between the peak area of the quantifier ion of the analyte and the peak area of the quantifier ion of the internal standard versus the corresponding concentrations of the analyte in concentration range between 0 and 100 ng/vial. Linearity was observed in the whole range. The values of the correlation factors R² of the calibration curves were higher than 0.99. The LOQ values obtained was 0.5 ng/vial calculated by Multiquant software 2.1 at an accuracy between 80-120% and CV %<20%. Recovery from different biological matrices with the two extraction methods ranged from 63 to 70

### Statistical Analysis

The experiments were performed using MFAT e DMFAT samples from a total of 5 human donors investigated. Tests were generally run in triplicate and the reported data are expressed as mean±standard deviation (SD). If necessary, appropriate statistical tests were performed using GraphPad Software (GraphPad Inc., SanDiego, CA, USA). Statistical analysis was also performed with the Statistical Package for Social Science (SPSS version 13, IBM, NY, USA). Statistical differences were evaluated by the analysis of variance followed by Tukey-Kramer multiple comparison test and by the two-tailed, unpaired Student test. p≤0.5 was considered statistically significant.

### Results

### PTX Displayed Anti-Proliferative Activity on Hep-3B Cell Line In Vitro

Initial experiments were performed to establish the efficacy of PTX to inhibit Hep-3B proliferation

To this end, different concentration of PTX (from 0.1 ng/ml to 1000 ng/ml) were added to culture medium. The dose of PTX required to reduce 50% (IC₅₀) and 90% (IC₉₀) Hep-3B growth was 15±2 and 25±8 ng/ml, respectively (FIG. 7). Thus, Hep-3B cells showed a significant resistance to PTX anti-proliferative activity when compared to other cancer cell lines previously tested (6, 14).

MFAT and DMFAT Specimens Loaded with PTX Exerted a Potent Antitumor Activity on Hep-3B In Vitro

The activity of CM derived from control and MFAT or DMFAT loaded with different concentration of PTX (0.05 to 4 μg/ml) was investigated (FIG. 1). CM derived from both cultured MFAT and DMFAT primed with 0.25 to 4.0 μg PTX showed a similar potent Hep-3B growth inhibition. However, DMFAT seemed more effective than MFAT particularly with a lower dosage of PTX (0.1 μg) (FIG. 1A). This trend was confirmed by evaluating PTX p-EC released in the medium. As shown in FIG. 1B, both DMFAT-PTX-CM and MFAT-PTX-CM showed a dose-dependent efficacy but p-EC resulted 12±2 and 18±5 ng/ml for MFAT and DMFAT respectively. The morphological appearance of Hep-3B cells treated with DMFAT-PTX-CM is shown in FIG. 8.

The anti-tumor activity of MFAT-PTX and DMFAT-PTX on Hep-3B was also studied by using trans-well inserts. A potent inhibition of Hep-3B proliferation that correlate with the increasing amount of PTX uploaded was obtained (FIG. 1C). No significant differences were observed between MFAT-PTX and DMFAT-PTX specimens among the different preparations in terms of efficacy. Besides the significant reduction in cancer cell number, the co-culture with DMFAT-PTX specimens induced many Hep-3B cells to acquire necrotic and apoptotic features (FIG. 9).

A significant inhibition of Hep-3B proliferation was also obtained by reducing to 1:4 (12.5 ul) the amount of MFAT-PTX and DMFAT-PTX specimens (uploaded with 2 μg of drug) seeded in trans-well inserts (FIG. 1D). DMFAT-PTX were enough effective to produce around 90% of Hep-3B growth inhibition and a trend of greater effectiveness than the MFAT-PTX was noted among AT preparations.

3D Matrigel Construct to Study Interaction Between Hep-3B Cells and DMFAT Loaded or not with PTX

To mimic an in vivo situation, 3D constructs by mixing control and DMFAT-PTX with Hep-3B cells, were prepared and then processed for H&E staining (FIG. 2). DMFAT showed the typical adipose tissue structure but appeared looser and more disaggregated in the control group (FIG. 2A) and in those loaded with PTX 0.5 mg/ml (FIG. 2B) if compared to specimens loaded at the maximal dose of PTX 1 mg/ml (FIG. 2C). At higher magnifications it was possible to observe the homogeneous distribution of Hep-3B cells inside the trabecular/spongy-like structure of the DMFAT specimens, apparently not showing any difference among control (FIG. 2D) and PTX loading (FIG. 2E, F).

However, the analysis of cancer cell apoptosis detected with Hoechst 33342 staining showed very few cells in control DMFAT sections (FIG. 2G) while in all DMFAT-PTX groups they were significantly increased (FIG. 2H, I). The apoptotic figures (chromatin condensation, nuclear fragmentation) were around 5 to 10-fold higher than those present in control DMFAT. The apoptotic effect of DMFAT-PTX on Hep-3B was also investigated through Annexin V expression and was significantly higher in DMFAT-PTX versus DMFAT and untreated cells as control, dependent on PTX dose (FIG. 2J) and not on the increasing number of Hep-3B cells placed in the 3D constructs (FIG. 2K). Similar results were obtained with fresh MFAT-PTX (data not shown).

### Anti-Tumor Effect of DMFAT-PTX in an HCC-Established Subcutaneous Growing Tumor

To verify the HCC nature of Hep-3B cell line, few mice were injected sc with 5×10⁶ Hep-3B cells and when the tumor nodule was palpable mice were sacrificed, tumor removed and analyzed by IHC for expression of HCC markers. All the tumor nodules analyzed showed an intense cellularity composed of large size cells, with several atypical mitoses. The hepatocellular nature of the cellularity was confirmed by the diffuse, strong positivity of immunostaining for Anti-Human Hepatocyte-hepar, Arginase and CK-PAN (FIG. 10).

The potential anti-cancer activity of a single-shot DMFAT-PTX administration at the tumor site as described under Materials and Methods (FIG. 11) was then evaluated. Under this schedule of treatments, control mice reached a tumor volume of around 2 cm³ in around 30-40 days (FIG. 3A). A similar tumor growth behavior was observed in mice treated locally with free drug PTX (FIG. 3B) and DMFAT (FIG. 3C). In contrast, mice treated with DMFAT-PTX (10 mg/kg) showed a significant delay of tumor growth. None of the mice reached 2 cm³ in tumor volume even at the maximal time of observation 60 days. Tumor was 0.904 0.312 cm³ that was around half volume of those of control and 33% of mice were even tumor free (FIG. 3D). However, using this high PTX dosages, in all treated mice skin lesions with a necrotic appearance were observed. None of the mice died and such lesions healed spontaneously without the need of any particular pharmacological treatment in around 2-3 weeks, in all cases (FIG. 12). Due to this observation, another group of mice was treated by reducing the concentration of DMFAT-PTX to 5 mg/Kg. At this dose none of the mice showed skin ulceration at the site of DMFAT-PTX injection and tumor growth was still significantly delayed, reaching around 2 cm³ volume after 58 days. At 60 days 15% of mice was tumor free (FIG. 3E). The trend of tumor growth in all groups of mice is shown in FIG. 3F; Table 1 summarizes all results.

TABLE 1

| | | | HCC (TW) | Day of | % of mice | % of mice |
|---|---|---|---|---|---|---|
| | | Groups | (Grams ± SD) | sacrifice | tumor-free | cured |
| | | CTRL | 1.793 ± 0.789 | 32 ± 3 | 0 | 0 |
| | | (saline) | | | | |
| | | DMFAT 200 μl | 2.125 ± 0.589 | 40 ± 7 | 0 | 0 |
| A | | DMFAT-PTX | 0.904 ± 0.312** | 60 ± 0** | 33** | 33** |
| | {open oversize bracket} | 200 μl/200 μg | | | | |
| | | (10 mg/kg) | | | | |
| | | PTX 200 μg | 2.010 ± 0.377 | 33 ± 3 | 0 | 0 |
| | | (10 mg/kg) | | | | |
| | | CTRL | 2.124 ± 0.560 | 34 ± 5 | 0 | 0 |
| B | {open oversize bracket} | DMFAT-PTX | 2.787 ± 0.643 | 58 ± 2** | 15 | 15 |
| | | 200 μl/100 μg | | | | |
| | | (5 mg/kg) | | | | |

Note:
in the mice receiving DMFAT-PTX 10 mg/Kg tumor weight was significantly reduced at 60 days after injection (**p < 0.01), in addition around 33% of mice were tumor-free and after 90 days were sacrificed and considered cured. Less efficacy was observed in mice treated with DMFAT-PTX loaded with half of the dose of PTX (5 mg/Kg). However, in this group, tumor weight was not significantly reduced compare to CTRL but growth was still significantly delayed (**p < 0.01 versus CTRL). In addition, 15% of mice were tumor-free.

No significant effect was observed in the group of mice treated with either DMFAT or PTX alone compared to CTRL.

To ascertain that treated mice were tumor-free, the animals that survived after 90 days were sacrificed and the tumor inoculation area was investigated. The histology of the skin tissue confirmed the absence of any residual neoplastic tissue, with disclosure of normal skin with minimal reactive areas of inflammation (FIG. 13).

Finally, the histological investigation of the tumor from control and treated mice was performed at the time of mice sacrifice. IHC did not show significant differences in the expression of HCC markers. The CK-PAN, Arginase and Anti-hepato positive cells were similarly present and staining was diffuse in control as well as in PTX and DMFAT treated tumors with except of DMFAT-PTX treated mice where Arginase staining resulted to be more focal (FIG. 4). Interestingly, in the tumor of mice injected with control DMFAT some residual fat tissue was still present at the time of mice sacrifice. This was not seen in the tumor of mice treated with DMFAT-PTX (at both dosages used), suggesting an accelerated digestion of the xenotransplanted adipose tissue primed with PTX (FIG. 14).

Significant differences were seen by investigating and quantifying Mib-1/Ki67 proliferative marker indexes. Indeed, a significant reduction of Ki67 expression in tumor of mice treated with DMFAT-PTX at both dosages was noted. In particular, in tumors treated at higher PTX dosages the number of Ki67 positive cells/field were almost 5 times lower (127±43 versus 654±123 of the control group). Some reduction of Ki67 expression was also noted in mice treated with free PTX drug (FIG. 5).

PK of PTX Delivered by DMFAT-PTX when Located Nearby Tumor Nodule

The PK of free PTX has been widely studied elsewhere (15, 16). Here we focused on investigating PK of PTX delivered by DMFAT when located nearby cancer cells (FIG. 6). To this end, a group of mice were injected with Hep-3B cancer cells and, when the tumor nodule was formed, DMFAT loaded with the reduced dosage of PTX 5 mg/kg (corresponding to 100 μg/200 ul, to avoid eventual skin ulcerations) was locally injected. A group of control, normal mice (no tumor injected) were similarly treated. In these group of normal mice PTX blood concentration at 2 hours was 200±34 ng/ml, that decreased to 22.4±5 at 24 hours, 17.1±3 at 72 hours and 6.8±2 ng/ml at 168 hours. (FIG. 6A). Although technical difficulties in recovering all sc DMFAT-PTX injected, the analysis of the residual amount of drug at the site of injection after 2 hours was 207±14 μg/g (about 41% of the injected amount of 500 μg/g). This concentration decreased significantly to 101±11 (24 hours), 13.2±3 (48 hours) and 5.2±0.3 μg/gr at 72 hours. At 7 days 1.14±0.1 μg/gr of PTX was found a local drug concentration that can be considered of pharmacological importance, since higher than the IC₅₀ of Hep-3B cancer cells (FIG. 6B). In summary, these data are similar to those previously reported using another strain of mice (6).

PTX release kinetics in the presence of the tumor demonstrated significant differences compared to normal mice. After 2 hours PTX in blood was around 90 ng/ml that went down to around 7 ng/ml at 24 hours, showing that in the blood of mice bearing tumors, PTX declined more rapidly. In addition, at 72 hours, PTX in blood was below a detectable level (FIG. 6C). At 2 hours after injection the DMFAT-PTX implanted near tumor nodule was, again, technically very difficult to recover completely, however the concentration of PTX in the residual DMFAT-PTX was significantly lower if compared to those obtained from normal mice and it was 86.58 μg/gr (only 17.3% of those initially injected) and nearby tumor nodule was undetectable. In the tumor, PTX detection started after 24 hours. At that time, we found that in residual DMFAT and in tumor PTX was 3.35 ug/gr and 0.41 ug/gr, respectively. At 48 hours, in DMFAT the concentration of PTX was reduced of about 50% (1.81 μg/gr), but in tumor increased 4 times (1.6 μg/gr), thus reaching a concentration of PTX almost equivalent to that of the adjacent DMFAT. In the subsequent analysis performed on days 3 and 7, PTX concentration in the tumor and in the adjacent DMFAT remained almost equivalent (FIG. 6D). The kinetics of PTX concentration in tumor and adipose tissue are summarized in FIGS. 6E and F. To note, local PTX concentrations present in both DMFAT and the tumor at 7 day were 0.2±0.08 and 0.30±0.18 μg/gr, respectively, resulting around 5 and 3-fold lower to that of recovered in normal mice (1.14±0.1 ug/gr). However, both PTX concentrations were still higher than the IC₅₀ of the PTX (0.015 μg/gr) on Hep-3B cells.

In summary, these data seem to indicate that tumor nodule initially accelerated the PTX release from DMFAT when located nearby. The equilibrium of PTX concentration in DMFAT and tumor was proximally reached in about 48 hours and, in the tumor, a concentration of PTX was significantly detectable for at least 7 days; this concentration must be considered of pharmacological importance.

### DISCUSSION

One of the main issues of systemic chemotherapy is its un-specificity, affecting both cancer cells and normal healthy ones, thus producing undesired side effects (17, 18). Therefore, developing new chemotherapy approaches that may act preferentially at the tumor site is of great interest to improve anti-cancer efficacy and quality of a cancer patient life (19, 20). With this aim, the authors of the present invention previously demonstrated that mesenchymal stromal cells (MSCs) can be an optimal tool to delivery anti-cancer drugs such as PTX and Doxorubicine (14, 21, 22).

The optimal ability of MSCs to act as scaffold cells and because adipose tissue (AT) is a natural container of MSCs, it was hypothesized that MFAT a derivative from liposuction would work even better to delivery drug, given its histological and structural scaffold-like characteristics (9, 10), The validity of this hypothesis was recently fully demonstrated by showing the capacity of both MFAT and DMFAT of incorporating and releasing PTX and when DMFAT-PTX was located in the area of tumor resection, it was able to block or delay cancer relapse (6).

In order to expand these results, it was investigated within the present invention whether a single-shot treatment of DMFAT-PTX located nearby tumor mass would be also effective in inhibiting the growth of a well-established primary HCC growing tumor in mice. HCC is a tumor for which systemic therapy is considered poor effective (23, 24, 25). The HCC features of the Hep-3B cell line here used was firstly confirmed by histological analysis of the tumor nodule formed after sc cells injection in nude mice.

Preliminary in vitro experiments to establish the sensitivity of Hep-3B cells to PTX inhibition revealed an IC₅₀ higher if compared to other tumor cell lines previously investigated (6, 14, 21). However, the in vitro activity of MFAT and DMFAT loaded with different concentration of PTX on Hep-3B. showed that both MFAT and DMFAT loaded with PTX were equally effective in inhibiting Hep-3B proliferation; either the co-culture or CM were very effective and was PTX priming dose dependent. These results were similar to those previously observed using other cancer cell lines (6).

By using a 3D assay that consisted in mixing Hep-3B cells with DMFAT specimens loaded or not with PTX, embedded in a Matrigel matrix to mimic an in vivo situation, the inventors demonstrated that PTX released by DMFAT caused a potent cancer cells apoptosis that was directly proportional to the dose of PTX loaded. The apoptotic effect was also confirmed by the analysis of Annexin V expression on Hep-3B and demonstrated that PTX is a cancer cell apoptotic inducer (26).

Although Hep-3B cells have been widely used in vivo to induce HCC tumors (27), the inventors performed a preliminary experiment to establish the dose of Hep-3B necessary to obtain 100% of tumor takes and timing to get a palpable tumor of 0.5-0.7 cm in diameter (around 100 mg in weight) in all mice. When the tumors appeared as a clearly palpable mass, DMFAT specimens loaded or not with PTX were placed as close as possible nearby cancer nodule. At this regard, intra-tumor type of injection, that would have been the optimal route of treatment, was not technically feasible due to the impossibility to inject 200 μl (200 mm³ volume) of DMFAT in a smaller tumor volume. (100-150 mm³). The PTX dose of 10 mg/kg initially used in this study was based on our previous work and it was near maximal drug concentration that can be loaded in 1 ml DMFAT and it was based on clinical studies in humans as well (6, 28). DMFAT-PTX treatment produced a potent tumor growth inhibition; at 60 days the tumor weight was around 1 g; in control or in mice treated either with free PTX or DMFAT the same tumor weight was reached only after 24-31 days and up to 2 g in about 29-36 days. In addition, 33% of DMFAT-PTX treated mice were even tumor free after 90 days post-transplant, as ascertained by histological examination of the skin area.

Notably, the high dose of PTX delivered by DMFAT caused an unwanted effect, producing in all mice a skin ulceration with formation of necrotic area at tumor site which, in any case, healed spontaneously without pharmacological treatments. Such aspect was not observed in our previous study where DMFAT-PTX were placed in the area of tumor resection or intraperitoneally injected (6). We think that this unwanted side effect, was caused because of the high dosage of PTX delivered and by the efficient drug concentration of DMFAT particularly when located in the subcutaneous area. Whatever the reason, this result clearly demonstrated the efficacy of DMFAT to restrain the drug mostly at the site of the injection. This conclusion was also supported by the following observations: 1) the local injection of free PTX drug, at similar high dosages, did not induce skin ulcers; 2) reducing the dose of drug uploaded in DMFAT did not induce skin ulcers, but anti-tumor activity was maintained. Finally, we analyzed in healthy and in tumor-bearing mice the PK of PTX released by DMFAT loaded with 5 mg/kg of PTX. In summary, in normal mice DMFAT retained most of PTX at local site of injection, blood PTX concentration decreased rapidly showing a PK similar to those previously observed but using a different healthy strain of mice (6). Instead, PTX delivered by DMFAT in tumor-bearing mice seemed to have a different PK. We noted that in blood PTX concentration decreased much rapidly; PTX was detectable only until 24 hours after subcutaneous DMFAT-PTX injection (in healthy mice until 7 days). Although technical difficulties in recovering all DMFAT-PTX injected particularly in tumor bearing mice, an accelerated PTX release, particularly at early time (2 hours), in the residual peritumoral fat tissue was observed. Only 14% of the total PTX initially injected was recovered (versus 40% in normal mice). In the adjacent tumor, PTX detection initiated at 24 hours with a kinetic that reached a drug concentration equilibrium between adipose and tumor tissue after 48 hours, remaining detectable in both tissues until 7 days. At this time, in the tumor the concentration of PTX was up to 10-fold higher its IC₅₀ and, therefore, it was consistent with the effect induced in vivo by DMFAT-PTX at same 5 mg/kg dose.

In conclusion results shown herein, taken together, strongly supported the initial hypothesis, and expanded previous results proposing MFAT and its derived devitalized DMFAT counterpart as a natural biomaterial able to absorb, transport and localized chemotherapeutic drugs.

This approach owes significant advantages compared to other scaffolds (natural/synthetic) potentially usable in the oncology field (29-32). In fact, beyond the easy availability of the material (the adipose tissue is easily obtainable by any patient through liposuction), the preparation of the MFAT is rapid and is carried out through a closed and sterile system (Lipogems® device) that does not require GMP conditions for clinical use (8). In addition, loading it with the drug is a fast procedure that can be carried out in the same operating room during surgery of patient with cancer, or prepared in advance and stored at −80 (DMFAT) before its preferentially autologous use.

Other studies are required to better understand the exact mechanism through which MFAT and DMFAT bind PTX (specifically, which lipid/proteins components are involved) and how the drug is released, if as free drug or through extracellular vesicles (33).

### REFERENCES

1) Raffetti E, Portolani N, Molfino S, Mentasti S, Baiocchi G L, Magoni M, et al. Is survival for hepatocellular carcinoma increasing? A population-based study on survival of hepatocellular carcinoma patients in the 1990s and 2000s. Clin Res Hepatol Gastroenterol 2021; 45(1):101433.
2) Bruix J, Sherman M. Management of hepatocellular carcinoma. Hepatology 2005; 42(5):1208-1236.
3) Perry J F, Charlton B, Koorey D J, Waugh R C, Gallagher P J, Crawford M D, et al. Outcome of patients with hepatocellular carcinoma referred to a tertiary centre with availability of multiple treatment options including cadaveric liver transplantation. Liver Int 2007; 27(9):1240-1248.
4) Portolani N, Coniglio A, Ghidoni S, Giovanelli M, Benetti A, Tiberio G A, et al. Early and late recurrence after liver resection for hepatocellular carcinoma: prognosis and therapeutic implications. Ann Surg 2006; 243(2):229-235.
5) Yang S I, Luo Y Y, Chen M, Zhou Y P, Lu F R, Deng D F, et al. A systematic review and meta-analysis comparing the prognosis of multicentric occurrence vs intrahepatic metastasis in patients with recurrent hepatocellular carcinoma after hepatectomy. HPB (Oxford) 2017; 19(10):835-842.
6) Alessandri G, Cocce V, Pastorino F, Paroni R, Dei Cas M, Restelli F, et al. Microfragmented human fat tissue is a natural scaffold for drug delivery: Potential application in cancer chemotherapy. J Control Release 2019 May 28; 302:2-18.
7) Tremolada C, Ricordi C, Caplan A I, Ventura C, Mesenchymal stem cells in Lipogems, a reverse story: from clinical practice to basic science, Methods Mol Biol 2016; 1416:109-122.
8) Tremolada C, Colombo V, Ventura C, Adipose tissue and mesenchymal stem cells:state of the art and Lipogems technology development, Curr Stem Cell Rep 2016; 2(3):304-312.
9) Ceserani V, Ferri A, Berenzi A, Benetti A, Ciusani E, Pascucci L, et al. Angiogenic and anti-inflammatory properties of micro-fragmented fat tissue and its derived mesenchymal stromal cells, Vasc Cell 2016; 8:3.
10) Nava S, Sordi V, Pascucci L, Tremolada C, Ciusani E, Zeira E, et al. Long Lasting Anti-Inflammatory Activity of Human Micro-Fragmented Adipose Tissue, Stem Cell International, 2019 Feb. 19; 2019:5901479.
11) Schiariti M P, Restelli F, Ferroli P, Benetti A, Berenzi A, Ferri A, et al. Fibronectin-adherent peripheral blood derived mononuclear cells as paclitaxel carriers for glioblastoma treatment: an in vitro study, Cytotherapy 2017; 19(6):721-734.
12) Berenzi A, Steimberg N, Boniotti J, Mazzoleni G, MRT letter: 3D culture of isolated cells: a fast and efficient method for optimizing their histochemical and immunocytochemical analyses, Microsc Res Tech 2015; 78(4):249-254.
13) Eisman J A, Barkla D, Tutton P J M, Suppression of the in vivo growth of human cancer solid tumor xenografts by 1,25-dihydroxyvitamin D3 Cancer Res 1987 1; 47(1):21-25.
14) Pessina A, Bonomi A, Cocce V, Invernici G, Navone S, Cavicchini L, et al. Mesenchymal stromal cells primed with paclitaxel provide a new approach for cancer therapy, PLoS One 2011; 6(12):e28321.
15) Clarke S J, Rivory L P, Clinical pharmacokinetics of docetaxel, Clin Pharmacokinet 1999; 36 (2):99-114.
16) Eiseman J L, Eddington N D, Leslie J, MacAuley C, Sentz D L, Zuhowski M, et al. Plasma pharmacokinetics and tissue distribution of paclitaxel in CD2F1 mice, Cancer Chemother Pharmacol 1994; 34(6):465-471.
17) Skeel R T, Khleif S N, Handbook of Cancer Chemiotherapy, Lippincott Williams & Wilkins, Philadelphia, 8^{th} edition 2011.
18) Barton-Burke M, Wilkes G M, Ingwersen K, Cancer Chemotherapy Care Plans Handbook, Jones & Bartlett learning, Sudbury, 3^{rd} edition, 2002.
19) Aigner K R, Stephens F O, Induction Chemotherapy: Integrated Treatment Programs for Locally Advanced Cancer, Springer Science & Business Media, New York, 2011.
20) Aigner K R, Gailhofer S, Kopp S, Regional versus systemic chemotherapy for advanced pancreatic cancer: a randomized study, Hepatogastroenterology 1998; 45(22):1125-1129.
21) Pessina A, Leonetti C, Artuso S, Benetti A, Dessy E, Pascucci L, et al., Drug releasing mesenchymal cells strongly suppress B16 lung metastasis in a syngeneic murine model, J Exp Clin Cancer Res 2015; 34(1):82.
22) Lisini D, Nava S, Frigerio S, Pogliani S, Maronati G, Marcianti A, et al. Automated Large-Scale Production of Paclitaxel Loaded Mesenchymal Stromal Cells for Cell Therapy Applications, Pharmaceutics 2020; 12(5):411.
23) Kang M K, Park J G, M D, Lee H J, Comparison of clinical outcomes between sorafenib and hepatic artery infusion chemotherapy in advanced hepatocellular carcinoma. A STROBE-compliant article, Medicine (Baltimore) 2018; 97(17):e0611.
24) Ikeda M, Morizane C, Ueno M, Okusaka T, Ishii H, Furuse J, Chemotherapy for hepatocellular carcinoma: current status and future perspectives, Japanese Journal of Clinical Oncology 2018; 48(2):103-114.
25) Schlageter M, Terracciano L M, D'Angelo S, Sorrentino P, Histopathology of hepatocellular carcinoma World J Gatroenterol 2014; 20(43):15955-15964.
26) Fan W, Possible mechanisms of paclitaxel-induced apoptosis, Biochemical Pharmacology 1999; 57(11):1215-1221.
27) Juang S H, Lung C C, Hsu P C, Hsu K S, Li Y C, Hong P C, et al. D-501036, a novel selenophene-based triheterocycle derivative, exhibits potent in vitro and in vivo antitumoral activity which involves DNA damage and ataxia telangiectasia-mutated nuclear protein kinase activation. Mol Cancer Ther 2007; 6(1):193-202.
28) Benguigui M, Alishekevitz D, Timaner M, Shechter D, Raviv Z, Benzekry S, et al. Dose- and time-dependence of the host-mediated response to paclitaxeltherapy: a mathematical modeling approach, Oncotarget 2018; 9:2574-2590.
29) Sousa I, Rodrigues F, Prazeres H, Lima R T, Soares P, Liposomal therapies in oncology: does one size fit all? Cancer Chemother Pharmacol 2018; 82(5):741-755.
30) Cox A, Andreozzi P, Dal Magro R, Fiordaliso F, Corbelli A, Talamini L, et al. Evolution of nanoparticle protein corona across the blood-brain barrier, ACS Nano 2018; 12:7292-7300.
31) Mahajan S, Patharkar A, Kuche K, Maheshwari R, Deb P K, Kalia K, et al. Functionalized carbon nanotubes as emerging delivery system for the treatment of cancer, Int J Pharm 2018; 548(1):540-558.
32) Krukiewicz K, Zak J K, Biomaterial-based regional chemotherapy: local anticancer drug delivery to enhance chemotherapy and minimize its side-effects, Mater Sci Eng C Mater Biol Appl 2016; 62:927-942.
33) Pascucci L, Cocce V, Bonomi A, Ami D, Ceccarelli P, Ciusani E, et al. Paclitaxel is incorporated by mesenchymal stromal cells and released in exosomes that inhibit in vitro tumor growth: a new approach for drug delivery, J Control Release 2014; 192:262-70.
34) Zeira O, Ghezzi E, Pettinari L, Re V, Lupi D M, Benali S L, et al. Case Report: Microfragmented Adipose Tissue Drug Delivery in Canine Mesothelioma: A Case Report on Safety, Feasibility, and Clinical Findings, Front Vet Sci 2021; 7:585427.

1-13. (canceled)

14. A method for the treatment and/or prevention of liver cancer, comprising administering an effective amount of a tissue-based drug delivery system comprising micro-fragmented fat tissue (MFAT) and an anticancer agent to a patient in need of such treatment or prevention.

15. The method of claim 14, wherein the liver cancer is hepatocellular carcinoma.

16. The method of claim 14, wherein the MFAT comprises clusters of fat tissue having size range from 10 to 5000 μm.

17. The method of claim 16, clusters of fat tissue having size range selected from the group consisting of:
a) from 100 to 3000 μm;
b) from 200 to 2500 μm;
c) from 30 to 1500 μm;
d) from 200 to 900 μm; and
e) from 400 to 900 μm.

18. The method of claim 14, wherein the MFAT is non-enzymatic micro-fragmented fat tissue.

19. The method of claim 14, wherein the MFAT is devitalized micro-fragmented fat tissue (DMFAT).

20. The method of claim 14, wherein the fat tissue is isolated from a mammalian source.

21. The method of claim 20, wherein the fat tissue is isolated from humans, said humans being alive or cadaver.

22. The method of claim 14, wherein the fat tissue is autologous or heterologous.

23. The method of claim 14, wherein the anticancer agent is selected from the group consisting of paclitaxel, docetaxel, lenvatenib gemcitabine, mitomycin C, vinorelbine, vincristin, vinblastin, nocodazole, epothilones, navelbine, teniposide, actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorethamine, mitomycin, mitoxantrone, nitrosourea, plicamycin, procarbazine, teniposide, triethylenethiophosphoramide and etoposide (VP16), adriamycin, amsacrine, camptothecin, daunorubicin, dactinomycin, doxorubicin, eniposide, epirubicin, etoposide, idarubicin, irinotecan (CPT-11) and mitoxantrone, pemetrexed, 5-fluorouracil (5-FU), metotrexate, cyclophosphamide, bortezomib, tomozolomide, sorafenib and combinations thereof.

24. The method of claim 23, wherein the anticancer agent is a combination of:
sorafenib and paclitaxel; or
cyclophosphamide and paclitaxel; or
adriamycin and paclitaxel.

25. The method of claim 14, wherein the amount of the anticancer agent ranges from 0.1 to 1 mg per ml of MFAT.

26. The method of claim 14, wherein the tissue-based drug delivery system is locally injected at the tumor tissue site and releases in situ a therapeutically effective amount of the anticancer agent.

27. The method of claim 23, wherein the anticancer agent is paclitaxel.

28. The method of claim 14, wherein the cancer is a liver primary or metastatic cancer.

29. The method of claim 28, wherein the cancer is primary or metastatic hepatocellular carcinoma.

30. The method of claim 14, further comprising administering a further therapeutic treatment.
